SEQUENCE LISTING (67 sequences)

SEQ ID NO: 1 - protein, 12 aa, Artificial Sequence
  Synthetic sequence DE12
EEEEEEDDDDDD

SEQ ID NO: 2 - protein, 18 aa, Artificial Sequence
  Synthetic sequence DE18
EEEEEEEEEDDDDDDDDD

SEQ ID NO: 3 - protein, 24 aa, Artificial Sequence
  Synthetic sequence DE24
EEEEEEEEEEEEDDDDDDDDDDDD

SEQ ID NO: 4 - protein, 30 aa, Artificial Sequence
  Synthetic sequence DE30
EEEEEEEEEEEEEEEDDDDDDDDDDDDDDD

SEQ ID NO: 5 - protein, 36 aa, Artificial Sequence
  Synthetic sequence DE36
EEEEEEEEEEEEEEEEEEDDDDDDDDDDDDDDDDDD

SEQ ID NO: 6 - protein, 88 aa, Artificial Sequence
  Synthetic sequence DED
NVEGKTGNATDEEEEEEEEEEEEDDDDDDDDDDDDEDSGAEIQDDDEEGFDDEEEFDDDDDDEHDDDDLENEENELEELEERVEARKK

SEQ ID NO: 7 - protein, 44 aa, Artificial Sequence
  Synthetic sequence DES
DLSNVEGKTGNATDEEEEEEEEEEEEDDDDDDDDDDDDEDSGAE

SEQ ID NO: 8 - protein, 24 aa, Artificial Sequence
  Synthetic sequence EO24
EEEEEEEEEEEEEEEEEEEEEEEE

SEQ ID NO: 9 - protein, 274 aa, Artificial Sequence
  Synthetic sequence NQ01
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 10 - protein, 546 aa, Artificial Sequence
  Synthetic luciferase sequence
EDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGK

SEQ ID NO: 11 - protein, 202 aa, Artificial Sequence
  Synthetic sequence HRV3C
DLVPRGSPEFPGRLERPHRDGPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQ

SEQ ID NO: 12 - protein, 298 aa, Artificial Sequence
  Synthetic sequence DE12-HRV3Csite-NQO1
MEEEEEEDDDDDDKGTLEVLFQGPMVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 13 - protein, 304 aa, Artificial Sequence
  Synthetic sequence DE18-HRV3Csite-NQO1
MEEEEEEEEEDDDDDDDDDKGTLEVLFQGPMVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 14 - protein, 310 aa, Artificial Sequence
  Synthetic sequence DE24-HRV3Csite-NQO1
MEEEEEEEEEEEEDDDDDDDDDDDDKGTLEVLFQGPMVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 15 - protein, 316 aa, Artificial Sequence
  Synthetic sequence DE30-HRV3Csite-NQO1
MEEEEEEEEEEEEEEEDDDDDDDDDDDDDDDKGTLEVLFQGPMVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 16 - protein, 322 aa, Artificial Sequence
  Synthetic sequence DE36-HRV3Csite-NQO1
MEEEEEEEEEEEEEEEEEEDDDDDDDDDDDDDDDDDDKGTLEVLFQGPMVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 17 - protein, 388 aa, Artificial Sequence
  Synthetic sequence DED-HRV3Csite-NQO1
MDYKDDDDKGMASDLSNVEGKTGNATDEEEEEEEEEEEEDDDDDDDDDDDDEDSGAEIQDDDEEGFDDEEEFDDDDDDEHDDDDLENEENELEELEERVEARKKASLEVLFQGPMVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 18 - protein, 341 aa, Artificial Sequence
  Synthetic sequence DES-HRV3Csite-NQO1
MDYKDDDDKGMASDLSNVEGKTGNATDEEEEEEEEEEEEDDDDDDDDDDDDEDSGAEASLEVLFQGPMVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 19 - protein, 321 aa, Artificial Sequence
  Synthetic sequence EO24-HRV3Csite-NQO1
MDYKDDDDKGMASEEEEEEEEEEEEEEEEEEEEEEEEASLEVLFQGPMVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 20 - protein, 386 aa, Artificial Sequence
  Synthetic sequence NQO1-HRV3Csite-DED
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARKGLEVLFQGPASDLSNVEGKTGNATDEEEEEEEEEEEEDDDDDDDDDDDDEDSGAEIQDDDEEGFDDEEEFDDDDDDEHDDDDLENEENELEELEERVEARKKASDYKDDDDK

SEQ ID NO: 21 - protein, 339 aa, Artificial Sequence
  Synthetic sequence NQO1-HRV3Csite-DES
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARKGLEVLFQGPASDLSNVEGKTGNATDEEEEEEEEEEEEDDDDDDDDDDDDEDSGAEASDYKDDDDK

SEQ ID NO: 22 - protein, 660 aa, Artificial Sequence
  Synthetic sequence DED-HRV3Csite-Luciferase
MDYKDDDDKGMASDLSNVEGKTGNATDEEEEEEEEEEEEDDDDDDDDDDDDEDSGAEIQDDDEEGFDDEEEFDDDDDDEHDDDDLENEENELEELEERVEARKKASLEVLFQGPEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGK

SEQ ID NO: 23 - protein, 613 aa, Artificial Sequence
  Synthetic sequence DES-HRV3Csite-Luciferase
MDYKDDDDKGMASDLSNVEGKTGNATDEEEEEEEEEEEEDDDDDDDDDDDDEDSGAEASLEVLFQGPEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGK

SEQ ID NO: 24 - protein, 535 aa, Artificial Sequence
  Synthetic sequence DED-HRV3C
MDYKDDDDKGMASDLSNVEGKTGNATDEEEEEEEEEEEEDDDDDDDDDDDDEDSGAEIQDDDEEGFDDEEEFDDDDDDEHDDDDLENEENELEELEERVEARKKMHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFPGRLERPHRDGPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQ

SEQ ID NO: 25 - DNA, 78 bp, Artificial Sequence
  Synthetic sequence DE12
gatgacaaaggtatggctagcgaggaggaagaggaagaagatgatgatgatgacgacgctagcctggaagttctgttc

SEQ ID NO: 26 - DNA, 78 bp, Artificial Sequence
  Synthetic sequence DE12
gaacagaacttccaggctagcgtcgtcatcatcatcatcttcttcctcttcctcctcgctagccatacctttgtcatc

SEQ ID NO: 27 - DNA, 30 bp, Artificial Sequence
  Synthetic sequence primer1
cgatacaaatggaaataataaccatctcgc

SEQ ID NO: 28 - DNA, 30 bp, Artificial Sequence
  Synthetic sequence primer2
cgcacagaatctaacgcttaataaatgtac

SEQ ID NO: 29 - DNA, 825 bp, Artificial Sequence
  Synthetic sequence NQ01
atggtgggacgccgtgctctgatcgtgctcgctcactcggaaagaacatcgttcaactacgctatgaaggaggctgctgccgctgccctgaagaagaagggctgggaggtggtcgaatccgacttgtacgctatgaacttcaaccccatcatctctcgtaaggacatcaccggcaagctgaaggatccagccaacttccagtacccggctgagtcagttttggcctacaaggaaggccacctgtcgcctgacatcgtggctgagcaaaagaagctcgaagctgccgatttggttatcttccagttccccttgcaatggttcggtgtgcctgctatcctgaagggctggttcgagagggtcttcatcggagaattcgcctacacttacgctgccatgtacgacaagggtccattcagatcgaagaaggccgtcctgtccatcaccactggtggcagcggatcaatgtacagcctccagggaatccacggtgacatgaacgtcatcctgtggccgatccaatctggcatcctccacttctgcggattccaggtgttggagccacaactgacatactccatcggacacaccccagctgacgctcgtatccagatcctcgaaggatggaagaagcgcttggagaacatctgggacgaaactcccttgtacttcgctccttcctctctgttcgatctcaacttccaggccggtttcctcatgaagaaggaggtccaagacgaggaaaagaacaagaagttcggcctgtctgttggacaccacctcggcaagagcatccccacagataaccagatcaaggctaggaagtaa

SEQ ID NO: 30 - DNA, 30 bp, Artificial Sequence
  Synthetic sequence primer3F
ctgttccagggtcccatggtgggacgccgt

SEQ ID NO: 31 - DNA, 36 bp, Artificial Sequence
  Synthetic sequence primer3R
ggagctcgaattcccttacttcctagccttgatctg

SEQ ID NO: 32 - DNA, 96 bp, Artificial Sequence
  Synthetic sequence DE18
gatgacaaaggtatggctagcgaagaagaggaggaggaagaggaagaagatgatgatgatgacgacgacgacgacgctagcctggaagttctgttc

SEQ ID NO: 33 - DNA, 96 bp, Artificial Sequence
  Synthetic sequence DE18
gaacagaacttccaggctagcgtcgtcgtcgtcgtcatcatcatcatcttcttcctcttcctcctcctcttcttcgctagccatacctttgtcatc

SEQ ID NO: 34 - DNA, 273 bp, Artificial Sequence
  Synthetic sequence DED
gatctaagtaatgtggaaggtaagacaggaaatgcaacagatgaagaggaggaagaagaggaggaggaagaggaagaagatgatgatgatgacgacgacgacgacgatgatgatgaagactctggagctgagatacaagatgatgatgaggaaggttttgatgatgaagaggaatttgatgatgacgatgatgatgaacatgatgatgatgatcttgagaatgaggaaaacgaactggaagagttggaagagagggtagaagccaggaagaaa

SEQ ID NO: 35 - DNA, 34 bp, Artificial Sequence
  Synthetic sequence primer4F
aatagatcttggtacccatggaagaggaggaaga

SEQ ID NO: 36 - DNA, 63 bp, Artificial Sequence
  Synthetic sequence primer4R
ggagctcgaattcccgggaccctggaacagaacttccagggtacctttatcatcatcgtcgtc

SEQ ID NO: 37 - DNA, 132 bp, Artificial Sequence
  Synthetic sequence DE30
gaacagaacttccaggctagcatcatcatcgtcgtcgtcgtcgtcatcatcatcatcgtcgtcgtcctcttcctcttcttcctcttcctcctcctcttcttcctcctcttcgctagccatacctttgtcatc

SEQ ID NO: 38 - DNA, 132 bp, Artificial Sequence
  Synthetic sequence DE30
gatgacaaaggtatggctagcgaagaggaggaagaagaggaggaggaagaggaagaagaggaagaggacgacgacgatgatgatgatgacgacgacgacgacgatgatgatgctagcctggaagttctgttc

SEQ ID NO: 39 - DNA, 150 bp, Artificial Sequence
  Synthetic sequence DE36
gaacagaacttccaggctagcatcatcatcgtcgtcgtcgtcgtcatcatcatcatcgtcgtcgtcgtcgtcgtcctcttcctcctcttcctcttcttcctcttcctcctcctcttcttcctcctcttcgctagccatacctttgtcatc

SEQ ID NO: 40 - DNA, 150 bp, Artificial Sequence
  Synthetic sequence DE36
gatgacaaaggtatggctagcgaagaggaggaagaagaggaggaggaagaggaagaagaggaagaggaggaagaggacgacgacgacgacgacgatgatgatgatgacgacgacgacgacgatgatgatgctagcctggaagttctgttc

SEQ ID NO: 41 - DNA, 36 bp, Artificial Sequence
  Synthetic sequence primer5F
caaaggtatggctagcgatctaagtaatgtggaagg

SEQ ID NO: 42 - DNA, 35 bp, Artificial Sequence
  Synthetic sequence primer5R
gaacttccaggctagctttcttcctggcttctacc

SEQ ID NO: 43 - DNA, 38 bp, Artificial Sequence
  Synthetic sequence primer6F
gacaaaggtatggctagcgatctaagtaatgtggaagg

SEQ ID NO: 44 - DNA, 37 bp, Artificial Sequence
  Synthetic sequence primer6R
ggaacagaacttccaggctagcctcagctccagagtc

SEQ ID NO: 45 - DNA, 114 bp, Artificial Sequence
  Synthetic sequence EO24
gatgacaaaggtatggctagcgaagaggaggaagaagaggaggaggaagaggaagaagaagaggaggaagaagaggaggaggaagaggaagaagctagcctggaagttctgttc

SEQ ID NO: 46 - DNA, 114 bp, Artificial Sequence
  Synthetic sequence EO24
gaacagaacttccaggctagcttcttcctcttcctcctcctcttcttcctcctcttcttcttcctcttcctcctcctcttcttcctcctcttcgctagccatacctttgtcatc

SEQ ID NO: 47 - DNA, 35 bp, Artificial Sequence
  Synthetic sequence primer7F
gttccagggtccagctagcgatctaagtaatgtgg

SEQ ID NO: 48 - DNA, 34 bp, Artificial Sequence
  Synthetic sequence primer7R
cgtccttgtagtcgctagctttcttcctggcttc

SEQ ID NO: 49 - DNA, 33 bp, Artificial Sequence
  Synthetic sequence primer8F
cctataaatagatctcccatggtgggacgccgt

SEQ ID NO: 50 - DNA, 36 bp, Artificial Sequence
  Synthetic sequence primer8R
gaacagaacttccagccccttcctagccttgatctg

SEQ ID NO: 51 - DNA, 39 bp, Artificial Sequence
  Synthetic sequence primer9F
gttccagggtccagctagcgatctaagtaatgtggaagg

SEQ ID NO: 52 - DNA, 37 bp, Artificial Sequence
  Synthetic sequence primer9R
catcgtccttgtagtcgctagcctcagctccagagtc

SEQ ID NO: 53 - DNA, 1638 bp, Artificial Sequence
  Synthetic luciferase sequence
gaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaag

SEQ ID NO: 54 - DNA, 30 bp, Artificial Sequence
  Synthetic sequence primer10F
ctgttccagggtcccgaagacgccaaaaac

SEQ ID NO: 55 - DNA, 35 bp, Artificial Sequence
  Synthetic sequence primer10R
ggagctcgaattcccttactttccgcccttcttgg

SEQ ID NO: 56 - DNA, 1296 bp, Artificial Sequence
  Synthetic sequence HRV3C
atgcaccaccaccatcatcattcgagcggtatgagccccattttggggtattggaaaatcaaaggtctggttcaaccaacccggctcctgcttgaatatcttgaagagaaatacgaagagcatctgtatgaacgtgacgaaggcgataaatggcgcaataagaagtttgaacttggcctggagtttccgaacttgccgtattacattgatggcgatgtgaaactgacacagtctatggcgattattcgctatattgcggacaaacacaacatgttaggcggttgcccgaaagaacgtgcggaaatctcaatgttagaaggggctgttctcgatattcgctatggcgtgtctcgtatcgcatacagtaaagactttgaaacgctgaaagtcgattttctttcgaaattgccggagatgctgaaaatgttcgaagatcggttgtgccacaaaacgtatctgaacggggatcatgtcacccatccggatttcatgttgtacgatgctctggatgtggtgctgtatatggacccaatgtgcttggacgcgtttccaaagctggtgtgtttcaagaaacgcattgaggccattccgcagattgataaatacctgaaaagctcgaaatatattgcgtggcctctgcagggttggcaagccacctttggtggcggagatcaccctccgaaaagcgatctggtcccgcgtgggagtcctgaatttccaggtcgccttgagcgcccgcatcgtgatggtccgaacacggaattcgcactgtccctcctgcgcaagaacattatgacaatcaccacgagcaaaggcgaattcactggactgggaatccatgatcgcgtgtgtgttattcccacccatgcacagcctggtgatgacgtcctggtaaatggccagaaaatccgcgttaaagacaaatacaaactggtagacccggaaaacatcaatctcgaactgaccgtgttaaccttagaccgtaacgagaaatttcgcgacattcgcggtttcatttccgaggacctcgaaggtgtggatgcaacgctggtagtgcattccaacaatttcacgaataccatcctggaagttggcccggttacaatggccggcttaatcaacctgtctagtactcccaccaatcgtatgattcgctatgattacgcgaccaagactggccaatgtggtggagtcttatgcgctactggcaaaatctttgggatccacgttggtggcaatggccgtcagggcttttcagcccaactgaagaaacagtacttcgtagaaaagcagtaa

SEQ ID NO: 57 - DNA, 42 bp, Artificial Sequence
  Synthetic sequence primer11F
cgatgacaaaggtatggctagcgatctaagtaatgtggaagg

SEQ ID NO: 58 - DNA, 39 bp, Artificial Sequence
  Synthetic sequence primer11R
gatgatggtggtggtgcattttcttcctggcttctaccc

SEQ ID NO: 59 - DNA, 39 bp, Artificial Sequence
  Synthetic sequence primer12F
gggtagaagccaggaagaaaatgcaccaccaccatcatc

SEQ ID NO: 60 - DNA, 44 bp, Artificial Sequence
  Synthetic sequence primer12R
ctcgaggagctcgaattcccttactgcttttctacgaagtactg

SEQ ID NO: 61 - DNA, 45 bp, Artificial Sequence
  Synthetic sequence primer13F
ctttaagaaggagatatacatatggactacaaggacgacgatgac

SEQ ID NO: 62 - DNA, 45 bp, Artificial Sequence
  Synthetic sequence primer13R
ccaccagtcatgctagccatatgttactgcttttctacgaagtac

SEQ ID NO: 63 - DNA, 16 bp, Artificial Sequence
  Synthetic sequence primer14
atgcgtccggcgtaga

SEQ ID NO: 64 - DNA, 18 bp, Artificial Sequence
  Synthetic sequence primer15
ccctcaagacccgtttag

SEQ ID NO: 65 - protein, 6 aa, Artificial Sequence
  Synthetic sequence DE6
EEEDDD

SEQ ID NO: 66 - DNA, 60 bp, Artificial Sequence
  Synthetic sequence DE6
gatgacaaaggtatggctagcgaggaagaagatgatgatgctagcctggaagttctgttc

SEQ ID NO: 67 - DNA, 60 bp, Artificial Sequence
  Synthetic sequence DE6
gaacagaacttccaggctagcatcatcatcttcttcctcgctagccatacctttgtcatc

(19) United States
(12) Patent Application Publication
(10) Pub. No.: US 2017/0291918 A1
(43) Pub. Date: Oct. 12, 2017

(54) METHOD FOR PURIFYING PROTEIN

(71) Applicant: SYSMEX CORPORATION, Kobe-shi (JP)

(72) Inventor: Ryo TAKAHASHI, Kobe (JP)

(73) Assignee: SYSMEX CORPORATION, Kobe-shi (JP)

(21) Appl. No.: 15/480,881

(22) Filed: Apr. 6, 2017

(30) Foreign Application Priority Data
Apr. 7, 2016 (JP) 2016-077127

(51) Int. Cl. C07K 1/18; C07K 14/00; C07K 7/08; C12P 21/02; C12N 9/02

(52) U.S. Cl. CPC C07K 1/18; C12P 21/02; C12N 9/0036; C12Y 106/05002; C07K 7/08; C07K 14/00; C12N 9/0069; C12Y 113/12007; C07K 2319/20; C07K 2319/50

(57) ABSTRACT

Disclosed is a method for purifying a protein, comprising steps of: preparing a sample containing a fusion protein containing an amino acid sequence of a peptide tag and an amino acid sequence of a target protein; and separating contaminant proteins contained with the fusion protein in the sample and the fusion protein in the sample, wherein the peptide tag contains 12 or more acidic amino acid residues.

### CROSS REFERENCE TO RELATED APPLICATIONS

This application claims priority from prior Japanese Patent Application No. 2016-077127, filed on Apr. 7, 2016, entitled “METHOD FOR PURIFYING PROTEIN, FUSION PROTEIN CONTAINING PEPTIDE TAG, AND PRODUCTION METHOD THEREOF”, the entire contents of which are incorporated herein by reference.

### TECHNICAL FIELD

The present invention relates to a method for purifying a protein.

### BACKGROUND

According to the progress of molecular biology, a system for mass expressing an objective protein by introducing a recombinant gene encoding the objective protein into a host such as E. coli, a yeast or a cell has been developed. In such system, a sample prepared from the host contains contaminant proteins such as host-derived endogenous proteins, together with the objective protein. Thus, the objective protein needs to be further purified. As a method for purifying an objective protein, for example, a method for purifying VP1 protein by introducing eight glutamic acids and cysteine into polyoma VP1 protein is disclosed in Stubenrauch K, et al, Purification of a viral coat protein by an engineered polyionic sequence. J. Chromatogr. B. Biomed. Sci. Appl: 737: 77-84, 2000.

### SUMMARY OF THE INVENTION

The scope of the present invention is defined solely by the appended claims, and is not affected to any degree by the statements within this summary.

A first aspect of the present invention is a method for purifying a protein. The method comprises steps of preparing a sample comprising a fusion protein and contaminant proteins, the fusion protein comprising an amino acid sequence of a peptide tag and an amino acid sequence of a target protein, and separating the contaminant proteins and the fusion protein. In the method, the peptide tag comprises 12 or more acidic amino acid residues.

A second aspect of the present invention is a method for purifying a protein, comprising steps of: preparing a sample comprising a fusion protein and contaminant proteins, the fusion protein comprising an amino acid sequence of a peptide tag and an amino acid sequence of a target protein, passing the sample through an anion exchange resin to obtain (i) the anion exchange resin to which the fusion protein is bound and (ii) a flow-through fraction comprising the contaminant proteins, removing the flow-through fraction, and passing a buffer through the anion exchange resin to elute the fusion protein from the anion exchange resin, a salt concentration of the buffer being 600 mM or more.

A third aspect of the present invention is a method for purifying a protein, comprising steps of: preparing a sample comprising a fusion protein and contaminant proteins, the fusion protein comprising an amino acid sequence of a peptide tag, an amino acid sequence of a target protein and a cleavable site recognized by a protease, the cleavable site being located between the amino acid sequence of the peptide tag and the amino acid sequence of the target protein, passing the sample through an anion exchange resin to obtain (i) the anion exchange resin to which the fusion protein is bound and (ii) a flow-through fraction comprising the contaminant proteins, removing the flow-through fraction, passing a buffer through the anion exchange resin to elute the fusion protein from the anion exchange resin to obtain a flow-through fraction comprising the fusion protein, a salt concentration of the buffer being 600 mM or more, mixing a protease recognizing the cleavable site and the flow-through fraction comprising the fusion protein to obtain a solution comprising a separated target protein and a separated peptide tag, and passing the solution comprising the separated target protein and the separated peptide tag through an anion exchange resin to obtain (i) the anion exchange resin to which the separated peptide tag is bound, and (ii) a flow-through fraction comprising the separated target protein.

### BRIEF DESCRIPTION OF THE DRAWINGS

FIGS. 1A to 1C are diagrams schematically showing a principle of the purification method of the present embodiment, in which FIG. 1A shows a separation step, and FIGS. 1B and 1C show a target protein acquisition step;

FIG. 2 is a figure showing the results of SDS (Sodium dodecyl sulfate)-polyacrylamide gel electrophoresis (SDS-PAGE) in Example 1;

FIG. 3 is a figure showing the results of SDS-PAGE in Example 2;

FIG. 4 is a figure showing the results of SDS-PAGE in Example 3;

FIG. 5 is a figure showing the results of SDS-PAGE in Example 4;

FIG. 6 is a figure showing the results of SDS-PAGE in Example 5;

FIG. 7 is a figure showing the results of SDS-PAGE in Example 6;

FIG. 8 is a figure showing the results of SDS-PAGE in Example 7;

FIG. 9 is a figure showing the results of SDS-PAGE in Example 8;

FIG. 10 is a figure showing the results of SDS-PAGE in Example 9;

FIG. 11 is a figure showing the results of SDS-PAGE in Example 10;

FIG. 12 is a figure showing the results of SDS-PAGE in Example 11;

FIG. 13 is a figure showing the results of SDS-PAGE in Example 12;

FIG. 14 is a figure showing the results of SDS-PAGE in Example 13;

FIG. 15 is a figure showing the results of SDS-PAGE in Comparative Example 1;

FIG. 16 is a figure showing the results of SDS-PAGE in Example 14;

FIG. 17 is a figure showing the results of SDS-PAGE in Example 15;

FIG. 18 is a figure showing the results of SDS-PAGE in Example 16;

FIG. 19 is a figure showing the results of SDS-PAGE in Example 17;

FIG. 20 is a figure showing the results of SDS-PAGE in Example 18;

FIG. 21 is a figure showing the results of SDS-PAGE in Example 19;

FIG. 22 is a figure showing the results of SDS-PAGE in Example 20;

FIG. 23 is a figure showing the results of SDS-PAGE in Example 21;

FIG. 24 is a figure showing the results of SDS-PAGE in Example 22;

FIG. 25 is a figure showing the results of SDS-PAGE in Example 23;

FIG. 26 is a figure showing the results of SDS-PAGE in Example 24;

FIG. 27 is a figure showing the results of SDS-PAGE in Example 25;

FIG. 28 is a figure showing the results of SDS-PAGE in Example 26;

FIG. 29 is a figure showing the results of SDS-PAGE in Example 27; and

FIG. 30 is a figure showing the results of SDS-PAGE in Example 28.

### DETAILED DESCRIPTION OF THE PREFERRED EMBODIMENTS

The purification method of the present embodiment includes steps of preparing a sample containing a fusion protein containing an amino acid sequence of a peptide tag and an amino acid sequence of a target protein and contaminant proteins (sample preparation step), and separating the fusion protein and the contaminant proteins (separation step). The contaminant protein means a protein such as host-derived endogenous proteins, other than the fusion protein and the target protein.

(Sample Preparation Step)

The fusion protein contains amino acid sequences of a peptide tag and a target protein. The peptide tag may be one containing 12 or more acidic amino acid residues. The number of the acidic amino acid residues in the peptide tag is preferably 18 residues or more, more preferably 24 residues or more, further preferably 30 residues or more, and particularly preferably 36 residues or more, since separation from the contaminant proteins is improved, and an effect on separation due to the isoelectric point of the target protein, pH of the buffer and the like can be more suppressed, in the separation step. The upper limit of the number of the acidic amino acid residues is not particularly limited. The upper limit of the number of the acidic amino acid residues is preferably 100 residues or less, and more preferably 70 residues or less, from the viewpoint of the effect on the three-dimensional structure of the fusion protein and the like. The acidic amino acid residues contained in the peptide tag may be either one of an aspartic acid residue or a glutamic acid residue or both of them. The peptide tag may contain a neutral amino acid residue and/or a basic amino acid residue, other than the acidic amino acid residue. The rate of the number of the acidic amino acid residues to the number of the amino acid residues of the total peptide tags is preferably 20% or more and more preferably 60% or more, since separation from the contaminant proteins is improved. In the sequence of the total peptide tags, the amino acid sequence containing the acidic amino acid residues may be a sequence in which all acidic amino acid residues are continuous, or one or a plurality of neutral amino acid residues and/or basic amino acid sequences may be interposed between the acidic amino acid residues. The isoelectric point (pI) of the peptide tag is preferably 5 or less and more preferably 4 or less, since separation from the contaminant proteins is improved, and the effects on separation due to the isoelectric point of the target protein, pH of the buffer and the like can be more suppressed. The isoelectric point is a pH value when the positive and negative ion concentrations in the aqueous solution become equal to each other. The isoelectric point can be measured, for example, by isoelectric electrophoresis, or the like. Specific examples of the peptide tag include the following peptide tags 1 to 8 (D; aspartic acid residue, E; glutamic acid residue).

### <Peptide Tag>

Peptide tag 1
(DE12; SEQ ID NO.: 1)
EEEEEEDDDDDD
Peptide tag 2
(DE18; SEQ ID NO.: 2)
EEEEEEEEEDDDDDDDDD
Peptide tag 3
(DE24; SEQ ID NO.: 3)
EEEEEEEEEEEEDDDDDDDDDDDD
Peptide tag 4
(DE30; SEQ ID NO.: 4)
EEEEEEEEEEEEEEEDDDDDDDDDDDDDDD
Peptide tag 5
(DE36; SEQ ID NO.: 5)
EEEEEEEEEEEEEEEEEEDDDDDDDDDDDDDDDDDD
Peptide tag 6
(DED; SEQ ID NO.: 6)
NVEGKTGNATDEEEEEEEEEEEEDDDDDDDDDDDDEDSGAEIQD
DDEEGFDDEEEFDDDDDDEHDDDDLENEENELEELEERVEARKK
Peptide tag 7
(DES; SEQ ID NO.: 7)
DLSNVEGKTGNATDEEEEEEEEEEEEDDDDDDDDDDDDEDSGAE
Peptide tag 8
(EO24; SEQ ID NO.: 8)
EEEEEEEEEEEEEEEEEEEEEEEE

The target protein is not particularly limited. Examples of the target protein include proteins such as enzymes, receptors, interferons, interleukins, antibodies, and fluorescent proteins. The isoelectric point of the target protein is not particularly limited. Generally, the isoelectric point of a protein is 6 to 7, and even when any protein is targeted, the isoelectric point can be lowered by fusing a peptide tag, to an extent that the peptide tag can be sufficiently separated from the contaminant proteins.

In the fusion protein, the peptide tag may be bound to either N-terminal side or C-terminal side of the target protein. The peptide tag may be adjacently bound to the target protein, or other amino acid sequence may be inserted between the target protein and the peptide tag. For example, when an amino acid sequence of the cleavable site to be recognized by a protease is inserted between the amino acid sequences of the fusion protein and the peptide tag, it becomes easier to acquire a target protein by cleaving the peptide tag from the fusion protein. The isoelectric point of the fusion protein is not particularly limited. The isoelectric point of the fusion protein is preferably less than 6 and more preferably less than 5. The isoelectric point of many proteins is generally 6 to 7, thus when the isoelectric point of the fusion protein is less than 6, separation of the fusion protein from the contaminant proteins becomes easier, and purity of the fusion protein can be improved.

The sample containing a fusion protein and contaminant proteins can be acquired by introducing a polynucleotide containing a base sequence encoding a peptide tag and a base sequence encoding a target protein into a host cell and expressing the fusion protein in the host cell. Expression of the fusion protein can be performed by a known genetic engineering method. A polynucleotide encoding a peptide tag and a polynucleotide encoding a target protein can be acquired by a known method such as chemical synthesis. The acquired polynucleotides can be amplified according to a known gene amplification method using these as templates. The amplified polynucleotide and an expression vector are treated with a restriction enzyme, and bound using an appropriate DNA ligase, whereby a recombinant expression vector containing the polynucleotides each encoding a peptide tag and a target protein can be constructed. In the construction of the expression vector, the base sequence encoding a peptide tag may be arranged at the 5′ side or 3′ side of the base sequence encoding a target protein. The base sequence encoding a peptide tag and the base sequence encoding a target protein may be adjacently arranged, or a base sequence encoding a cleavable site recognized by a protease or the like may be inserted therebetween. The host is not particularly limited. As the host, E. coli, a yeast, an insect cell, an insect body, an animal cell, a plant cell or the like can be used. The expression vector is not particularly limited. The expression vector includes plasmid vectors, pharge vectors, virus vectors and the like. The expression vector can be properly selected depending on the used host. The expression vector may contain a regulatory sequence such as a replication origin, a promoter sequence or an enhancer sequence, or a sequence such as a selectable marker. Introduction of an expression vector into a host can be performed according to a known method depending on the host. Examples of the method include a calcium phosphate method, an electroporation method, a lipofection method, and the like. As described above, a transformant in which the expression vector is introduced into the host cell can be obtained. The obtained transformant is incubated under preferred conditions, thus a fusion protein can be produced.

After expressing the fusion protein, treatment such as extraction, crushing, centrifugation, solubilization and body fluid collection is performed on cells of the host, tissue, insect body, and the like, as necessary, to acquire a sample as a cell culture liquid, an extract, a homogenate, a solution, a body fluid, or the like. The sample contains contaminant proteins such as host-derived endogenous proteins, other than the produced fusion protein. For the preparation of the sample, a buffer such as a phosphate buffer, a citrate buffer, an acetate buffer, a tris buffer, a MOPS buffer or a HEPES buffer can be used. These buffers contain a salt with a weak acid or weak base used for obtaining a buffer action. In addition, salts such as sodium chloride, potassium chloride and calcium chloride can be used. The salt concentration of a buffer or sample herein means a concentration of a salt other than the salt intended for buffer action described above. The salt concentration of a sample is preferably 50 mM or more and 500 mM or less, and more preferably 50 mM or more and 250 mM or less, from the viewpoint of stability of the fusion protein, easiness of adjustment in the separation step, and the like. The pH of a sample is preferably 5 or more and 9 or less, and more preferably 6 or more and 8 or less, from the viewpoint of stability of the fusion protein, easiness of adjustment in the separation step, and the like.

When a sample is acquired using an Escherichia coli expression system, a plasmid vector can be used as an expression vector. Specific examples of the plasmid vector include pET, pGEX, pCold, pMAL, pCAL, and the like. The expression vector may contain a promoter sequence such as a replication origin, lac, T7 or tac, an enhancer sequence, or a sequence of a selectable marker such as ampicillin resistance gene or kanamycin resistance gene. Introduction of an expression vector into E. coli can be performed by a calcium phosphate method, an electroporation method, a lipofection method, and the like. After the introduction of an expression vector, E. coli is cultured in a medium containing the selectable marker, whereby a transformant in which the expression vector is introduced into E. coli can be selected. The thus-obtained transformant is proliferated under preferred conditions. Then, the proliferated transformant is expressed and induced depending on the selected promoter to produce a fusion protein. After expressing the fusion protein, a sample containing the fusion protein and contaminant proteins derived from E. coli can be acquired by crushing E. coli or the like.

When using an insect body such as silkworm or insect cells as an expression system, a transfer vector can be used. The transfer vector is transduced into the insect cells together with baculovirus DNA, and then an objective gene can be inserted into the baculovirus DNA by homologous recombination. Examples of the transfer vector include pM01, pM02, pHS01, pHS02, and the like. The transfer vector preferably contains a promoter such as a polyhedrin promoter or a p10 promoter. Examples of the insect cells include Sf9, Sf21, High5, TN-368, Bm5, and the like. Examples of the baculovirus include nuclear polyhedrosis virus (NPV), and specifically, AcMNPV, BmNPV and the like can be exemplified. The transfer vector and linearized baculovirus DNA are introduced into the insect cells, whereby homologous recombination occurs, and a recombinant baculovirus into which an objective gene is inserted is obtained. A virus solution containing a recombinant baculovirus is inoculated into an insect body or insect cells, or the like, whereby the recombinant baculovirus can be infected. The insect body such as silkworm may be in any form of imago, pupa, and larva. The insect body or insect cells are infected with the recombinant baculovirus, and bred or cultured for 1 to 7 days, whereby a fusion protein can be expressed. After expressing the fusion protein, a sample containing the fusion protein and contaminant proteins derived from the insect body or insect cells can be acquired by crushing the insect body or insect cells, or the like.

(Fusion Protein)

The fusion protein in the present embodiment can be obtained, as the sample preparation step described above, by a production method including steps of introducing a polynucleotide containing a base sequence encoding an amino acid sequence of a peptide tag and a base sequence encoding an amino acid sequence of a fusion protein into a host cell, and expressing the fusion protein in the host cell. The fusion protein contains the amino acid sequence of a peptide tag containing 12 or more acidic amino acid residues and the amino acid sequence of a target protein. Examples of the fusion protein of the present embodiment include the following constructs 1 to 12 obtained by fusing NAD(P)H dehydrogenase, quinone 1 human (NQO1; pI=8.91; SEQ ID NO.: 9) or Luciferase (pI=6.71; SEQ ID NO.: 10) that is a target protein and the above peptide tags 1 to 8, and inserting a cleavable site recognized by protease HRV3C therebetween. The following construct 13 obtained by fusing HRV3C (pI=8.46; SEQ ID NO.: 11) that is a target protein and the above peptide tag 7 is also exemplified.

Construct 1 DE12-HRV3Csite-NQO1 (SEQ ID NO.: 12)

Construct 2 DE18-HRV3Csite-NQO1 (SEQ ID NO.: 13)

Construct 3 DE24-HRV3Csite-NQO1 (SEQ ID NO.: 14)

Construct 4 DE30-HRV3Csite-NQO1 (SEQ ID NO.: 15)

Construct 5 DE36-HRV3Csite-NQO1 (SEQ ID NO.: 16)

Construct 6 DED-HRV3Csite-NQO1 (SEQ ID NO.: 17)

Construct 7 DES-HRV3Csite-NQO1 (SEQ ID NO.: 18)

Construct 8 EO24-HRV3Csite-NQO1 (SEQ ID NO.: 19)

Construct 9 NQO1-HRV3Csite-DED (SEQ ID NO.: 20)

Construct 10 NQO1-HRV3Csite-DES (SEQ ID NO.: 21)

Construct 11 DED-HRV3Csite-Luciferase (SEQ ID NO.: 22)

Construct 12 DES-HRV3Csite-Luciferase (SEQ ID NO.: 23)

Construct 13 DED-HRV3C (SEQ ID NO.: 24)

(Separation Step)

As to the sample containing the fusion protein obtained in the sample preparation step and the contaminant proteins, the fusion protein and the contaminant proteins are separated. The phrase “the fusion protein and the contaminant proteins are separated” includes extracting and purifying the fusion protein, and storing into a container different from the contaminant proteins. The contaminant proteins may be substantially completely separated, or a part of the contaminant proteins may be separated. A part or all of the contaminant proteins is separated, whereby the purity of the fusion protein can be improved.

The separation means is not particularly limited, and a known separation means can be used. Separation by an ion exchange resin is preferred, and separation by an anion exchange resin is more preferred, since good separation from the contaminant proteins is obtained. The ion exchange group of the anion exchange resin is not particularly limited, and a quaternary ammonium (QA), a quaternary aminoethyl (QAE), diethylaminoethyl (DEAE) and the like can be used. Specific examples include Hitrap HP Q (GE Healthcare), TOYOPEARL GigaCap Q-650M, TOYOPEARL Q-600C AR, TOYOPEARL QAE-550, TOYOPEARL DEAE-650M, TOYOPEARL SuperQ-650M (all, Tosoh Corporation), Q101, DE101 (Mitsubishi Chemical Corporation), and the like. As a carrier of the ion exchange group, cellulose, dextran, agalose, a hydrophilic vinyl polymer or the like can be used. The sample containing the fusion protein obtained in the sample preparation step and the contaminant proteins is passed through such anion exchange resin to bind the fusion protein to the anion exchange resin. Thereafter, for example, the salt concentration of an eluate is increased to elute the contaminant proteins, then the fusion protein is eluted from the ion exchange resin with the eluate with a high salt concentration, whereby a purified fusion protein can be acquired. Examples of a method for increasing the salt concentration of an eluate include a stepwise method in which the salt concentration is stepwisely changed to elute an objective protein, a gradient method in which the salt concentration is continuously changed to elute an objective protein, and the like. As a buffer used for elution, a buffer such as a phosphate buffer, a citrate buffer, an acetate buffer, a tris buffer, a MOPS buffer or a HEPES buffer can be used. The salt concentration of the buffer is preferably set in the range of 50 mM or more and 2000 mM or less, and more preferably in the range of 50 mM or more and 1000 mM or less, from the viewpoint of separation from the contaminant proteins, stability of the fusion protein, and the like. Particularly, when the salt concentration of the buffer is set at 600 mM or more and more preferably 750 mM or more, separation from the contaminant proteins is improved. The range of pH of the buffer is not particularly limited. The range of pH of the buffer is preferably in the range of 5 or more and 9 or less, and more preferably in the range of 6 or more and 8 or less, from the viewpoint of separation from the contaminant proteins, stability of the fusion protein, and the like.

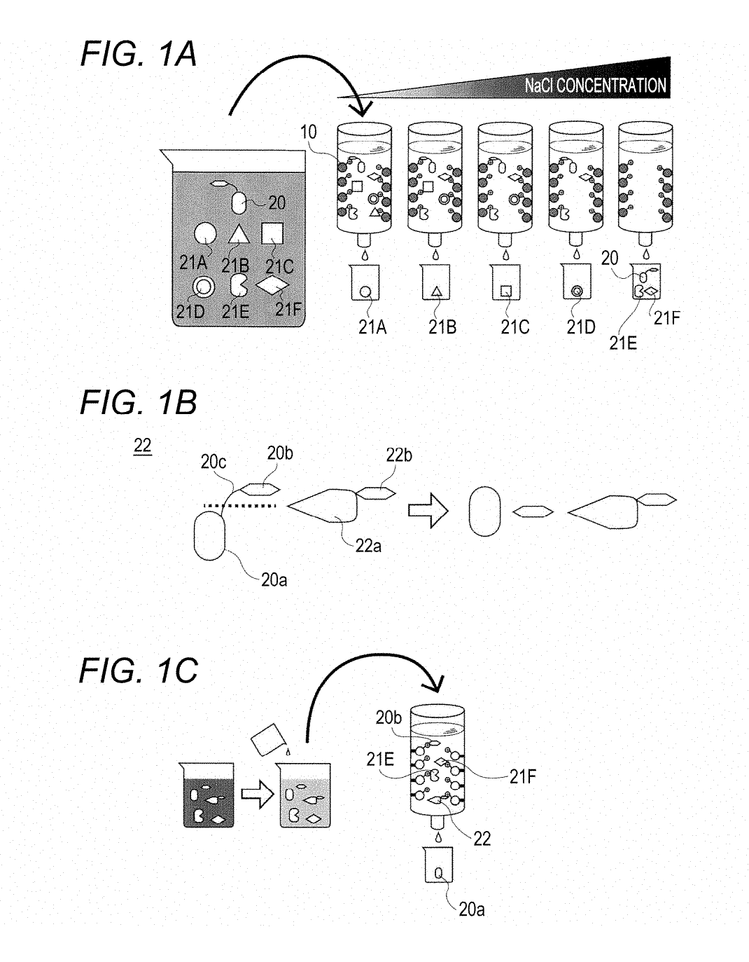

FIGS. 1A to 1C are schematic diagrams showing one embodiment, and FIG. 1A is a diagram showing a separation step. The principle of the separation step in the purification method of the present embodiment will be described based on this diagram. When a fusion protein is expressed in a host cell, a sample such as a disrupted cell suspension contains contaminant proteins 21A to 21F such as host-derived endogenous proteins, other than a fusion protein 20. The isoelectric points of the contaminant proteins 21 are high in the order of A to F. In the present embodiment, a peptide tag 20b containing 12 or more acidic amino acid residues is bound to a target protein 20a to lower the isoelectric point of the fusion protein 20. The fusion protein 20 in which the isoelectric point is lowered can be held in an anion exchange resin 10 up to a high salt concentration. On the other hand, most of host-derived contaminant proteins cannot be held in the anion exchange resin 10 up to the same salt concentration. The fusion protein and the contaminant proteins are separated, based on the difference in the isoelectric points. Specifically, when a sample is brought into contact with the anion exchange resin 10, the contaminant protein 21A with a high isoelectric point passes without binding to the anion exchange resin. Other contaminant proteins and the fusion protein 20 bind to the anion exchange resin 10. When the salt concentration of the eluate is increased, contaminant proteins are eluted in the higher order of the isoelectric point (21B to 21D). The fusion protein 20 is eluted by an eluate with further higher salt concentration. This elution fraction contains contaminant proteins 21E and 21F with an isoelectric point close to that of the fusion protein 20. The salt concentration of the eluate is adjusted as described above, whereby many contaminant proteins can be separated, thus a high purity fusion protein can be obtained. The difference in the isoelectric points between the fusion protein and the contaminant proteins is relatively large, thus good separation is obtained also by a stepwise method in which the salt concentration is stepwisely changed to elute a protein.

(Target Protein Acquisition Step)

The fusion protein and the contaminant proteins contained in the sample can be separated as described above. When the fusion protein further contains a cleavable site to be recognized by a protease, the protease is reacted with the separated fusion protein, whereby a peptide tag is cleaved from the fusion protein, and only a target protein can be acquired. The purification method of the present embodiment, after the separation step, may further include a step of cleaving the peptide tag from the fusion protein in the solution, using a protease as described above, to acquire a target protein.

The kind of the protease is not particularly limited. Examples include HRV3C, PreScission protease, factor Xa, thrombin, TEV protease, and the like. For example, such protease is added to the eluate containing a target protein obtained in the separation step, whereby the peptide tag can be cleaved from the fusion protein. The reaction temperature and the reaction time can be properly set according to the kind of the protease and the like. In the solution after reaction, the target protein, the peptide tag and the protease are contained. The target protein can be separated from this solution, using a known separation means. The separation means is not particularly limited, and a known separation means such as various chromatography can be used. Among them, the difference in the isoelectric points is caused between the target protein and the peptide tag when the peptide tag is cleaved from the fusion protein, thus separation by an ion exchange resin using the difference in the isoelectric points is preferred, and separation by an anion exchange resin is further preferred. When a solution containing the target protein, the peptide tag and the protease after reaction by the protease is brought into contact with the anion exchange resin, the cleaved peptide tag binds to the ion exchange resin, and the target protein passes without binding to the ion exchange resin. Therefore, the target protein can be acquired by collecting this flow-through fraction. The salt concentration of the solution containing the target protein, the peptide tag and the protease is preferably 500 mM or less and more preferably 300 mM or less, since separation from the peptide tag is improved. The pH of the solution is preferably 5 or more and 9 or less, and more preferably 6 or more and 8 or less. As a buffer used for adjustments of the salt concentration and pH of the solution, a phosphate buffer, a citrate buffer, an acetate buffer, a tris buffer, a MOPS buffer, a HEPES buffer or the like can be used. Even when contaminant proteins remain in the solution fraction containing a fusion protein obtained in the separation step, the isoelectric point of those contaminant proteins is relatively close to that of the fusion protein. Therefore, the difference in the isoelectric point from the remained contaminant proteins is also increased by cleaving the peptide tag from the fusion protein. Therefore, when using an ion exchange resin, separation from contaminant proteins is also improved, and a higher purity target protein can be obtained. The protease can be separated by a known separation means. When a difference is present in the isoelectric points of the target protein and the protease, both can be easily separated by an ion exchange resin.

The isoelectric point of the protease is preferably different from the isoelectric point of the target protein. This makes it possible to easily separate the target protein and the protease, after cleaving the peptide tag with the protease. The protease may be fused with a peptide tag. This makes it possible to adjust the isoelectric point of the protease to a desired value. As the peptide tag fused to the protease, the same one as the peptide tag to be bound to the fusion protein described above can be used. The amino acid sequence of the peptide tag to be bound to the protease may be the same as or different from the amino acid sequence of the peptide tag to be bound to the fusion protein. The amino acid sequence of the peptide tag to be bound to the protease is preferably the same as the amino acid sequence of the peptide tag contained in the fusion protein. Thereby, the protease has the isoelectric point almost same as that of the fusion protein and also different from that of the target protein. The protease fused with the peptide tag preferably does not have a cleavable site to be recognized by this protease, in order to avoid decomposition by itself. When the protease fused with the peptide tag is used, the target protein, the cleaved peptide tag and the protease fused with the peptide tag are contained in the solution after reaction. This solution is passed through an anion exchange resin, whereby the protease fused with the peptide tag also binds to the anion exchange resin, together with the cleaved peptide tag, thus a step of separating the protease is not separately required.

The protease fused with the peptide tag can be obtained by using the protease as the target protein, in the expression of the fusion protein described above. The fusion protein and the protease can be expressed by introducing a vector into which a polynucleotide containing a base sequence encoding both amino acid sequences into a host. A vector into which a polynucleotide encoding the fusion protein is introduced and a vector into which a polynucleotide encoding the protease is inserted may be introduced into the host to express the fusion protein and the protease. The vector into which a polynucleotide encoding the fusion protein is inserted is introduced into the host, and the vector into which a polynucleotide encoding the protease is inserted is introduced into the host, and the fusion protein and the protease are expressed alone respectively, and then may be mixed. In this case, the hosts may be the same kind or the different kind, and are preferably the same kind, in the aspect of easiness of handling after expression and the like. The isoelectric point of the protease fused with the peptide tag is not particularly limited. The isoelectric point is preferably less than 6 and more preferably less than 5, since separation from the target protein is improved.

Based on the FIGS. 1B and 1C, the principle of the target protein acquisition step in the purification method of the present embodiment will be described. The fusion protein 20 contains a cleavable site 20c to be recognized by the protease 22, between the target protein 20a and the peptide tag 20b. In a protease 22 fused with the peptide tag, a peptide tag 22b is bound to a protease 22a. When the fusion protein 20 and the protease 22 are reacted in the solution, the peptide tag 20b is cleaved from the fusion protein 20. The target protein 20a, the cleaved peptide tag 20b, the protease 22 fused with the peptide tag and contaminant proteins 21E and 21F remained in the separation step are contained in the solution after reaction. A peptide tag 20b with a low isoelectric point is cleaved, thus the isoelectric point of the target protein 20a becomes high as compared to that of the fusion protein 20. The contaminant proteins 21E and 21F have an isoelectric point relatively close to that of the fusion protein 20, thus the target protein 20a has an isoelectric point higher than those of those proteins. Furthermore, the target protein 20a has an isoelectric point higher than those of the cleaved peptide tag 20b and the protease 22 fused with the peptide tag 22b. Therefore, when this solution is diluted to lower the salt concentration, and passed through the anion exchange resin 10, the cleaved peptide tag 20b, the protease fused with the peptide tag 22 and contaminant proteins 21E and 21F bind to the anion exchange resin 10, but the target protein 20a passes through without binding thereto. High purity target protein 20a can be acquired by collecting this flow-through fraction.

### EXAMPLES

Hereinbelow, the present invention will be further described in detail with reference to examples, but the present invention is not limited to these examples.

### Example 1 [Expression and Purification of Fusion Protein DE12-HRV3Csite-NQO1 (Construct 1; pI=5.84)]

Peptide tag 1 (DE12) was fused to the N-terminal side of NAD(P)H dehydrogenase, quinone 1 human (NQO1; pI=8.91) that is a target protein, and expression and purification of a fusion protein containing a cleavable site HRV3Csite recognized by protease HRV3C between NQO1 and DE12 were performed.

(1) Construction of pM01_DE12 Vector

A pM01_DE12 vector in which the peptide tag DE12 was inserted into a pM01 vector was constructed.

Polynucleotides encoding DE12 (SEQ ID NOs.: 25 and 26) were annealed in pairs to prepare an insert part. A NheI site of the pM01 vector (SYSMEX CORPORATION) was treated with NheI (TAKARA BIO INC.), and the insert part was inserted using In-Fusion HD Cloning Kit (Clontech Laboratories, Inc.). Stellar Competent Cells were transformed with the constructed vector, then the obtained transformant was seeded on a LBA plate, and cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primer 1 (SEQ ID NO.: 27) and primer 2 (SEQ ID NO.: 28) for sequence confirmation, then the base sequence was analyzed by a DNA sequencer (Thermo Fisher Scientific Inc.). A vector into which the insert part was introduced, that had no variation in other part, was defined as pM01_DE12.

(2) Construction of pM01_DE12_HRV3Csite_NQO1

An expression vector pM01_DE12_HRV3Csite_NQO1 into which a polynucleotide (SEQ ID NO.: 29) encoding NQO1 (SEQ ID NO.: 9) was inserted into pM01_DE12 was constructed.

The polynucleotide encoding NQO1 and primers 3F (SEQ ID NO.: 30) and 3R (SEQ ID NO.: 31) corresponding to the insert part were prepared, and the insert part was amplified using KOD-Plus—(TOYOBO CO., LTD.) as a PCR enzyme, under the following PCR conditions. The amplified PCR product was migrated by 1.0% (w/v) agarose electrophoresis, and the part matched to the length of the insert part was cut out from the gel and purified to prepare an insert part. A SmaI site of the pM01_DE12 vector was treated with SmaI (TAKARA BIO INC.), and the insert part was inserted using In-Fusion HD Cloning Kit (Clontech Laboratories, Inc.). Stellar Competent Cells (Clontech Laboratories, Inc.) were transformed with the constructed vector, then the obtained transformant was seeded on a LBA plate. The transformant was cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primers 1 and 2 for sequence confirmation, then the base sequence was analyzed by a DNA sequencer (Thermo Fisher Scientific Inc.). A vector into which the insert part was introduced, that had no variation in other part, was defined as pM01_DE12_HRV3Csite_NQO1.

### <PCR Conditions>

Reaction 1: 94.0° C., 2-minute reaction 2: 94.0° C., 15-second reaction 3: 52.0° C., 30-second reaction 4: 68.0° C., 1-minute reaction 5: reaction 2 to reaction 4 are repeated 30 times per 1 kbp

(3) Preparation of Recombinant Baculovirus and Expression of Fusion Protein DE12-HRV3Csite-NQO1

pM01_DE12_HRV3Csite_NQO1 and baculovirus DNA were introduced into silkworm culture cells (BmN cells), and homologous recombination of virus was performed. The culture cells were cultured for 6 days, and the recombinant virus was collected. Thereafter, a virus solution diluted 50 times with MilliQ water was inoculated into pupa of silkworm, and the pupa was incubated for 6 days.

(4) Purification of DE12-HRV3Csite-NQO1

After incubation, the pupa of silkworm was collected, and 50 mL of a buffer (20 mM Tris-HCl (pH 8.0), 150 mM NaCl, 1 tablet Complete EDTA free (Roche), phenylthiourea) was added per a pupa to homogenate the pupa. The solution was centrifuged (8,000 g, 10 minutes), then the supernatant fraction was filtered with 0.8 uM of a filter agent, and the filtrate was collected. The collected filtrate was loaded on an ion exchange resin (Hitrap Q HP (1 mL)), and purified using a mixed solution of 20 mM Tris-HCl (pH 8.0, 150 mM NaCl; buffer A) and 20 mM Tris-HCl (pH 8.0, 1000 mM NaCl; buffer B). First, only buffer A (NaCl concentration of 150 mM) was flown through the ion exchange resin (EL0), and a fraction eluted with a 3:1 mixed solution of buffer A:buffer B (NaCl concentration of 363 mM) (EL1), a fraction eluted with a 1:1 mixed solution of buffer A:buffer B (NaCl concentration of 575 mM) (EL2), a fraction eluted with a 1:3 mixed solution of buffer A:buffer B (NaCl concentration of 788 mM) (EL3), and finally, a fraction eluted with only buffer B (NaCl concentration of 1000 mM) (EL4) were each collected.

(5) Detection of DE12-HRV3Csite-NQO1

SDS (Sodium dodecyl sulfate)-polyacrylamide gel electrophoresis (SDS-PAGE) was performed using 5 to 20% gradient gel and an electrophoresis apparatus (APRO Science Inc.). A sample and β-ME Sample Treatment for Tris SDS (COSMO BIO Co., Ltd. were mixed in a volume ratio of 1:1, and treated at 100° C. for 3 minutes, then 5 uL of the resulting solution was loaded on the gel. As a molecular weight marker, 2.5 uL of Blue Star (NIPPON Genetics Co., Ltd.) was loaded on the gel. The gel loaded with the sample was migrated at a voltage of 400 V for 14 minutes, and then transferred to a membrane using Trans-Blot Turbo Transfer System (Bio-Rad Laboratories, Inc.). The transferred membrane was set in i-Bind system (Thermo Fisher Scientific Inc.), and an antigen-antibody reaction was performed. In the antigen-antibody reaction, an ANTI-FLAG (registered trademark) M2-Peroxidase (HRP) antibody (Merck & Co., Inc.) or an Anti-NQO1 antibody (Cell Signaling Technology, Inc.) and Anti-IgG (H+L chain) (Mouse) pAb-HRP (Beckman Coulter, Inc.) were diluted 2000 times and used. Also, Luminata Forte (Merck & Co., Inc.) as a HRP reagent for detection and Gel Doc XR+system (Bio-Rad Laboratories, Inc.) as a detector were used.

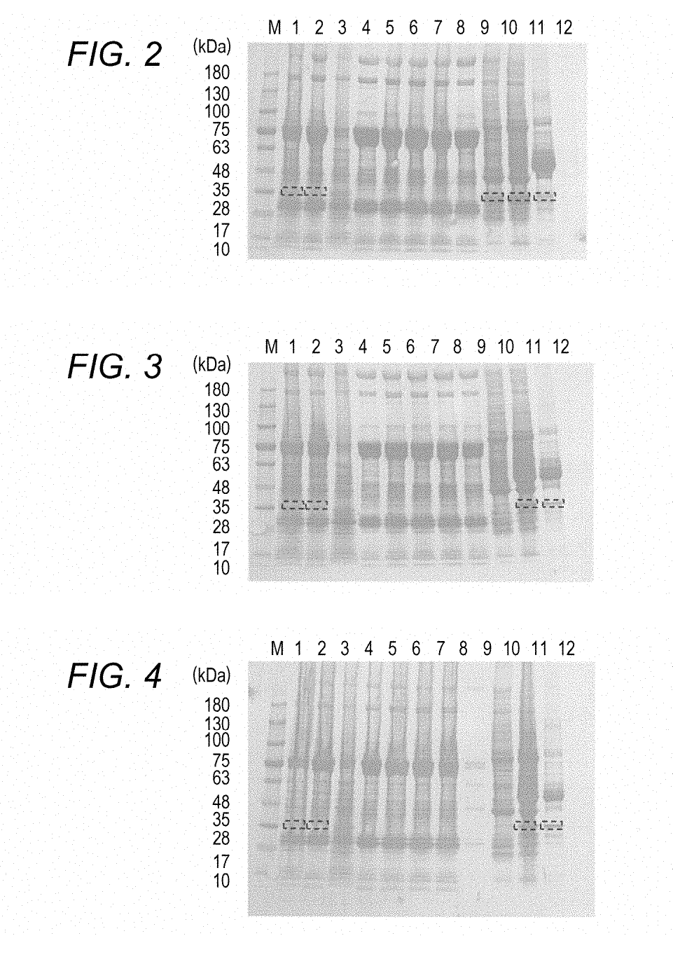

After performing western blotting, the used membrane was stained by GelCode Blue Stain Reagent (Thermo Fisher Scientific Inc.), and excess staining was bleached, then the image was photographed using Gel Doc XR+system (Bio-Rad Laboratories, Inc.). The results were shown in FIG. 2. In FIG. 2, the results of electrophoresis are shown as follows; M: molecular weight marker, lane 1: homogenate solution, lane 2: supernatant fraction after centrifugation, lane 3: precipitated fraction after centrifugation, lanes 4 to 7: flow-through fractions 1 to 4 of the ion exchange resin, lane 8: elution fraction by buffer A (NaCl concentration of 150 mM) (EL0), lane 9: elution fraction by a 3:1 mixed solution of buffer A:buffer B (NaCl concentration of 363 mM) (EL1), lane 10: elution fraction by a 1:1 mixed solution of buffer A:buffer B (NaCl concentration of 575 mM) (EL2), lane 11: elution fraction by a 1:3 mixed solution of buffer A:buffer B (NaCl concentration of 788 mM) (EL3), and lane 12: elution fraction by buffer B (NaCl concentration of 1000 mM) (EL4). The part surrounded by a broken line shows a band containing the target protein. Explanation of each lane is common in FIGS. 3 to 15.

### Example 2 [Expression and Purification of Fusion Protein DE18-HRV3Csite-NQO1 (Construct 2; pI=5.03)]

Peptide tag 2 (DE18) was fused to the N-terminal side of NQO1 that is a target protein, and expression and purification of a fusion protein containing HRV3Csite between NQO1 and DE18 were performed.

The same procedures were carried out as in Example 1, except for preparing an insert part using polynucleotides (SEQ ID NOs.: 32 and 33) encoding DE18 (SEQ ID NO.: 2) in place of the polynucleotides encoding the peptide tag DE12, to construct a pM01_DE18 vector.

Further, pM01_DE18_HRV3Csite_NQO1 was constructed, and a recombinant baculovirus was prepared in the same manner as in Example 1, then expression, purification and detection of fusion protein DE18-HRV3Csite-NQO1 were performed. The results were shown in FIG. 3.

### Example 3 [Expression and Purification of Fusion Protein DE24-HRV3Csite-NQO1 (Construct 3; pI=4.78)]

Peptide tag 3 (DE24) was fused to the N-terminal side of NQO1 that is a target protein, and expression and purification of a fusion protein containing HRV3Csite between NQO1 and DE24 were performed.

(1) Construction of pM01_DE24 Vector

A pM01_DE24 vector in which a polynucleotide encoding peptide tag DE24 was inserted into a pM01 vector was constructed as below.

The polynucleotide (SEQ ID NO.: 34) encoding peptide tag 6 (DED; SEQ ID NO.: 6) and primers 4F (SEQ ID NO.: 35) and 4R (SEQ ID NO.: 36) corresponding to the insert part were prepared, and the insert part was amplified using KOD-Plus—(TOYOBO CO., LTD.) as a PCR enzyme, under the same PCR conditions as in Example 1. The amplified PCR product was migrated by 1.0% (w/v) agarose electrophoresis, and the part matched to the length of the insert part was cut out from the gel and purified to prepare an insert part. A SmaI site of the pM01 vector (SYSMEX CORPORATION) was treated with SmaI (TAKARA BIO INC.), and the insert part was inserted using In-Fusion HD Cloning Kit (Clontech Laboratories, Inc.). Stellar Competent Cells (Clontech Laboratories, Inc.) were transformed with the constructed vector, then the obtained transformant was seeded on a LBA plate. The transformant was cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primers 1 and 2 for sequence confirmation, then the base sequence was analyzed by a DNA sequencer (Thermo Fisher Scientific Inc.). A vector into which the insert part was introduced, that had no variation in other part, was defined as pM01_DE24.

(2) pM01_DE24_HRV3Csite_NQO1 was constructed, and a recombinant baculovirus was prepared in the same manner as in Example 1, then expression, purification and detection of fusion protein DE24-HRV3Csite-NQO1 were performed. The results were shown in FIG. 4.

### Example 4 [Expression and Purification of Fusion Protein DE30-HRV3Csite-NQO1 (Construct 4; pI=4.61)]

Peptide tag 4 (DE30) was fused to the N-terminal side of NQO1 that is a target protein, and expression and purification of a fusion protein containing HRV3Csite between NQO1 and DE30 were performed.

The same procedures were carried out as in Example 1, except for preparing an insert part using polynucleotides (SEQ ID NOs.: 37 and 38) encoding DE30 (SEQ ID NO.: 4) in place of the polynucleotides encoding the peptide tag DE12, to construct a pM01_DE30 vector.

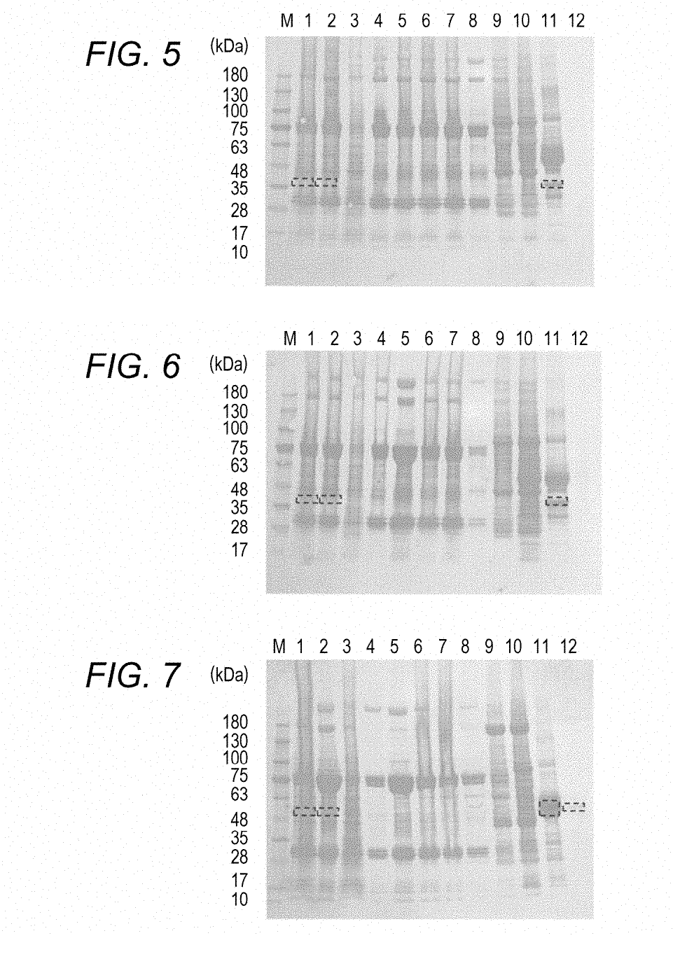

Further, pM01_DE30_HRV3Csite_NQO1 was constructed, and a recombinant baculovirus was prepared in the same manner as in Example 1, then expression, purification and detection of fusion protein DE30-HRV3Csite-NQO1 were performed. The results were shown in FIG. 5.

### Example 5 [Expression and Purification of Fusion Protein DE36-HRV3Csite-NQO1 (Construct 5; pI=4.49)]

Peptide tag 5 (DE36) was fused to the N-terminal side of NQO1 that is a target protein, and expression and purification of a fusion protein containing HRV3Csite between NQO1 and DE36 were performed.

The same procedures were carried out as in Example 1, except for preparing an insert part using polynucleotides (SEQ ID NOs.: 39 and 40) encoding DE36 (SEQ ID NO.: 5) in place of the polynucleotides encoding the peptide tag DE12, to construct a pM01_DE36 vector.

Further, pM01_DE36_HRV3Csite_NQO1 was constructed, and a recombinant baculovirus was prepared in the same manner as in Example 1, then expression, purification and detection of fusion protein DE36-HRV3Csite-NQO1 were performed. The results were shown in FIG. 6.

### Example 6 [Expression and Purification of Fusion Protein DED-HRV3Csite-NQO1 (Construct 6; pI=4.26)]

Peptide tag 6 (DED) was fused to the N-terminal side of NQO1 that is a target protein, and expression and purification of a fusion protein containing HRV3Csite between NQO1 and DED were performed.

(1) Construction of pHS01_DED Vector

A pHS01_DED vector in a peptide tag DED (SEQ ID NO.: 6) was inserted into a pHS01 vector was constructed as below.

The polynucleotide (SEQ ID NO.: 34) encoding the peptide tag DED and primers 5F (SEQ ID NO.: 41) and 5R (SEQ ID NO.: 42) corresponding to the insert part were prepared, and the insert part was amplified using KOD-Plus—(TOYOBO CO., LTD.) as a PCR enzyme, under the same PCR conditions as in Example 1. The amplified PCR product was migrated by 1.0% (w/v) agarose electrophoresis, and the part matched to the length of the insert part was cut out from the gel and purified to prepare an insert part. A NheI site of the pHS01 vector (SYSMEX CORPORATION) was treated with NheI (TAKARA BIO INC.), and the insert part was inserted using In-Fusion HD Cloning Kit (Clontech Laboratories, Inc.). Stellar Competent Cells were transformed with the constructed vector, then the obtained transformant was seeded on a LBA plate. The transformant was cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primers 1 and 2 for sequence confirmation, then the base sequence was analyzed by a DNA sequencer (Thermo Fisher Scientific Inc.). A vector into which the insert part was introduced, that had no variation in other part, was defined as pHS01_DED.

(2) HS01_DED_HRV3Csite_NQO1 was constructed, and a recombinant baculovirus was prepared in the same manner as in Example 1, then expression, purification and detection of fusion protein DED-HRV3Csite-NQO1 were performed. The results were shown in FIG. 7.

### Example 7 [Expression and Purification of Fusion Protein DES-HRV3Csite-NQO1 (Construct 7; pI=4.55)]

Peptide tag 7 (DES) was fused to the N-terminal side of NQO1 that is a target protein, and expression and purification of a fusion protein containing HRV3Csite between NQO1 and DES were performed.

The same procedures were carried out as in Example 6, except for using primers 6F (SEQ ID NO.: 43) and 6R (SEQ ID NO.: 44) as the primers corresponding to the insert part, to construct pHS01_DES.

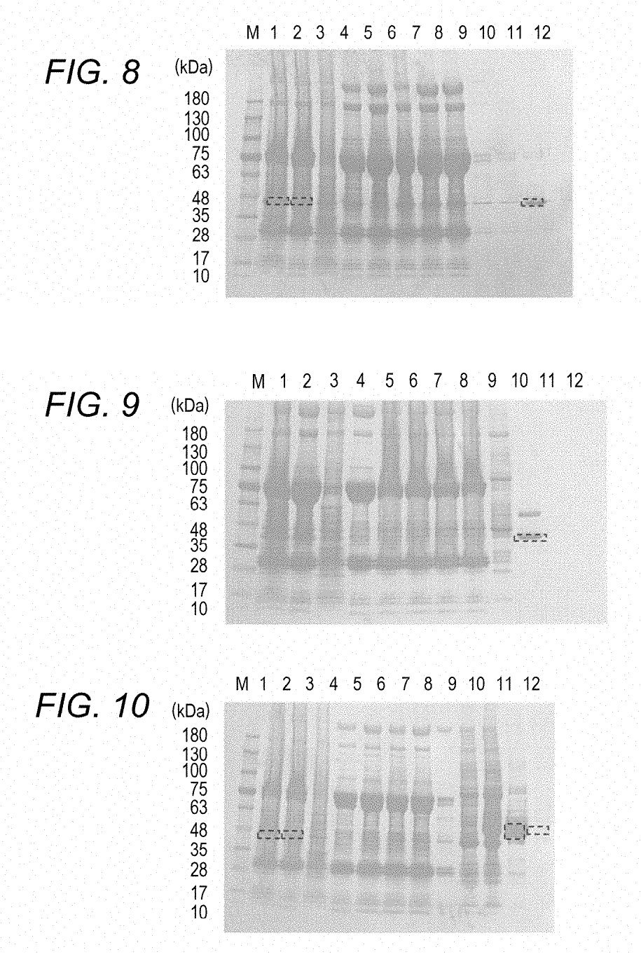

pHS01_DES_HRV3Csite_NQO1 was constructed, and a recombinant baculovirus was prepared in the same manner as in Example 1, then expression, purification and detection of fusion protein DES-HRV3Csite-NQO1 were performed. The results were shown in FIG. 8.

### Example 8 [Expression and Purification of Fusion Protein EO24-HRV3Csite-NQO1 (Construct 8; pI=4.73)]

Peptide tag 8 (EO24) was fused to the N-terminal side of NQO1 that is a target protein, and expression and purification of a fusion protein containing HRV3Csite between NQO1 and EO24 were performed.

(1) Construction of pHS01_EO24

Polynucleotides encoding peptide tag EO24 (SEQ ID NOs.: 45 and 46) were annealed in pairs to prepare an insert part. A NheI site of the pHS01 vector (SYSMEX CORPORATION) was treated with NheI (TAKARA BIO INC.), and the insert part was inserted using In-Fusion HD Cloning Kit (Clontech Laboratories, Inc.). Stellar Competent Cells (Clontech Laboratories, Inc.) were transformed with the constructed vector, then the obtained transformant was seeded on a LBA plate. The transformant was cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primers 1 and 2 for sequence confirmation, then the base sequence was analyzed by a DNA sequencer (Thermo Fisher Scientific Inc.). A vector into which the insert part was introduced, that had no variation in other part, was defined as pHS01_EO24.

(2) pHS01_EO24_HRV3Csite_NQO1 was constructed, and a recombinant baculovirus was prepared in the same manner as in Example 1, then expression, purification and detection of fusion protein EO24-HRV3Csite-NQO1 were performed. The results were shown in FIG. 9.

### Example 9 [Expression and Purification of Fusion Protein NQO1-HRV3Csite-DED (Construct 9; pI=4.26)]

The peptide tag DED was fused to the C-terminal side of NQO1 that is a target protein, and expression and purification of a fusion protein containing HRV3Csite between NQO1 and DED were performed.

(1) Construction of pHS02 DED Vector

pHS02_DE12 Vector in which a polynucleotide encoding the peptide tag DED was inserted into pHS02 vector was constructed as below.

The polynucleotide (SEQ ID NO.: 34) encoding the peptide tag DED and primers 7F (SEQ ID NO.: 47) and 7R (SEQ ID NO.: 48) corresponding to the insert part were prepared, and the insert part was amplified using KOD-Plus—(TOYOBO CO., LTD.) as a PCR enzyme, under the same PCR conditions as in Example 1. The amplified PCR product was migrated by 1.0% (w/v) agarose electrophoresis, and the part matched to the length of the insert part was cut out from the gel and purified to prepare an insert part. A NheI site of the pHS02 vector (SYSMEX CORPORATION) was treated with NheI (TAKARA BIO INC.), and the insert part was inserted using In-Fusion HD Cloning Kit (Clontech Laboratories, Inc.). Stellar Competent Cells (Clontech Laboratories, Inc.) were transformed with the constructed vector, then the obtained transformant was seeded on a LBA plate. The transformant was cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primers 1 and 2 for sequence confirmation, then the base sequence was analyzed by a DNA sequencer (Thermo Fisher Scientific Inc.). A vector into which the insert part was introduced, that had no variation in other part, was defined as pHS02_DED.

(2) The same procedures were carried out as in Example 1, except for using primers 8F (SEQ ID NO.: 49) and 8R (SEQ ID NO.: 50) as the primers corresponding to the insert part, to construct pHS02_NQO1_HRV3Csite_DED.

(3) Further, a recombinant baculovirus was prepared in the same manner as in Example 1, and expression, purification and detection of fusion protein NQO1-HRV3Csite-DED were performed. The results were shown in FIG. 10.

### Example 10 [Expression and Purification of Fusion Protein NQO1-HRV3Csite-DES (Construct 10; pI=4.55)]

Peptide tag DES was fused to the C-terminal side of NQO1 that is a target protein, and expression and purification of a fusion protein containing HRV3Csite between NQO1 and DES were performed.

(1) The same procedures were carried out as in Example 9, except for using 9F (SEQ ID NO.: 51) and 9R (SEQ ID NO.: 52) as the primers, to construct pHS02_DES.

(2) The same procedures were carried out as in Example 9 to construct pHS01_NQO1_HRV3Csite_DES.

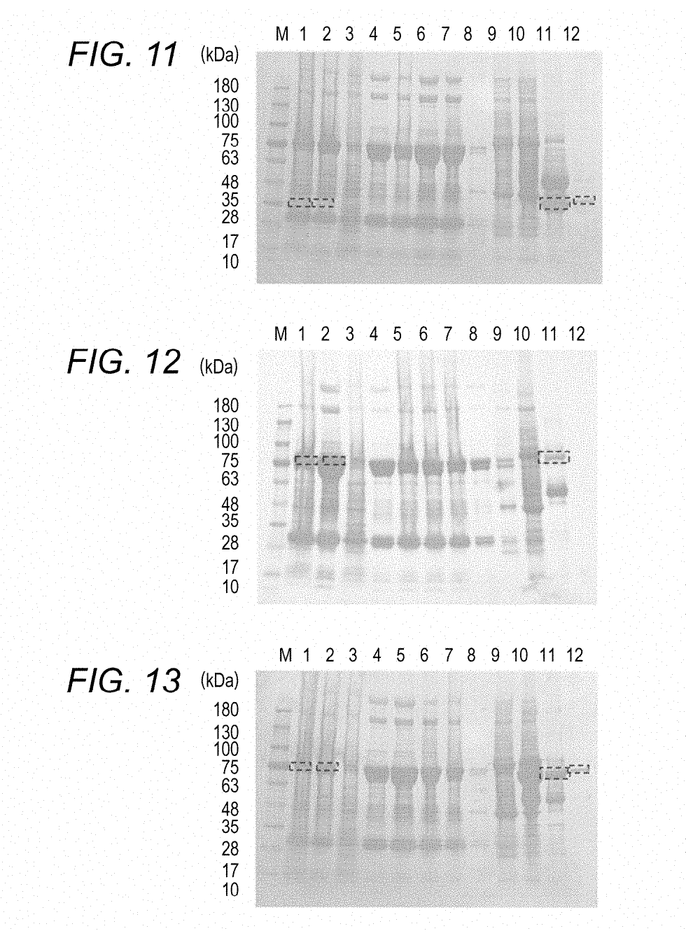

(3) A recombinant baculovirus was prepared in the same manner as in Example 1, and expression, purification and detection of fusion protein NQO1-HRV3Csite-DES were performed. The results were shown in FIG. 11.

### Example 11 [Expression and Purification of Fusion Protein DED-HRV3Csite-Luciferase (Construct 11; pI=4.47)]

(1) Construction of pHS01_DED Vector

The same procedures were carried out as in Example 6 to construct pHS01_DED.

(2) Construction of pHS01_DED_HRV3Csite_Luciferase

The polynucleotide (SEQ ID NO.: 53) encoding Luciferase and primers 10F (SEQ ID NO.: 54) and 10R (SEQ ID NO.: 55) corresponding to the insert part were prepared, and the insert part was amplified using KOD-Plus—(TOYOBO CO., LTD.) as a PCR enzyme, under the same PCR conditions as in Example 1. The amplified PCR product was migrated by 1.0% (w/v) agarose electrophoresis, and the part matched to the length of the insert part was cut out from the gel and purified to prepare an insert part. A SmaI site of the pHS01_DED vector was treated with SmaI (TAKARA BIO INC.), and the insert part was inserted using In-Fusion HD Cloning Kit (Clontech Laboratories, Inc.). Stellar Competent Cells (Clontech Laboratories, Inc.) were transformed with the constructed vector, then the obtained transformant was seeded on a LBA plate. The transformant was cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primers 1 and 2 for sequence confirmation, then the base sequence was analyzed by a DNA sequencer (Thermo Fisher Scientific Inc.). A vector into which the insert part was introduced, that had no variation in other part, was defined as pHS01_DED_HRV3Csite_Luciferase.

(3) A recombinant baculovirus was prepared in the same manner as in Example 1, and expression, purification and detection of fusion protein DED-HRV3Csite-Luciferase were performed. The results were shown in FIG. 12.

### Example 12 [Expression and Purification of Fusion Protein DES-HRV3Csite-Luciferase (Construct 12; pI=4.75)]

The same procedures were carried out as in Example 11, except for using a pHS01_DES vector constructed in Example 7 in place of the pHS01_DED vector, to construct pHS02_DES_HRV3Csite_Luciferase.

A recombinant baculovirus was prepared in the same manner as in Example 1, and expression, purification and detection of fusion protein DES-HRV3Csite-Luciferase were performed. The results were shown in FIG. 13.

### Example 13 [Expression and Purification of Fusion Protein DED-HRV3C (Construct 13; pI=4.75)]

(1) Construction of pET17b_DED_HRV3C

The polynucleotide (SEQ ID NO.: 34) encoding the peptide tag DED, a polynucleotide (SEQ ID NO.: 56) encoding HRV3C, primers 11F and 11R (SEQ ID NOs.: 57 and 58; DED) corresponding to the insert part and primers 12F and 12R (SEQ ID NOs.: 59 and 60; HRV3C) were prepared, and the insert part was amplified using KOD-Plus—(TOYOBO CO., LTD.) as a PCR enzyme, under the same PCR conditions as in Example 1. The amplified PCR product was migrated by 1.0% (w/v) agarose electrophoresis, and the part matched to the length of the insert part was cut out from the gel and purified to prepare an insert part. A SmaI site and a NheI site of the pHS01 vector (SYSMEX CORPORATION) were treated with SmaI (TAKARA BIO INC.) and NheI (TAKARA BIO INC.), and the two insert parts were inserted using In-Fusion HD Cloning Kit (Clontech Laboratories, Inc.). Stellar Competent Cells (Clontech Laboratories, Inc.) were transformed with the constructed vector, then the obtained transformant was seeded on a LBA plate. The transformant was cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primers 1 and 2 for sequence confirmation, then the base sequence was analyzed by a DNA sequencer (Thermo Fisher Scientific Inc.). A vector into which the insert part was introduced, that had no variation in other part, was defined as pHS01_DED_HRV3C.

Next, an insert for pET17b was prepared. Primers 13F (SEQ ID NO.: 61) and 13R (SEQ ID NO.: 62) were prepared, and the primers were amplified using pHS01_DED_HRV3C as a template, and using KOD-Plus—(TOYOBO CO., LTD.) as a PCR enzyme, under the same PCR conditions as in Example 1. The amplified PCR product was migrated by 1.0% (w/v) agarose electrophoresis, and the part matched to the length of the insert part was cut out from the gel and purified to prepare an insert part. A NdeI site of the pET17b Vector (Novagen, Inc.) was treated with NdeI (TAKARA BIO INC.), and the insert part was inserted using In-Fusion HD Cloning Kit (Clontech Laboratories, Inc.). Stellar Competent Cells (Clontech Laboratories, Inc.) were transformed with the constructed vector, then the obtained transformant was seeded on a LBA plate. The transformant was cultured at 37° C. for 16 hrs. An ampicillin resistant transformant was selected from the obtained transformants according to normal procedure, and purification of plasmid was performed. In order to confirm a base sequence of the selected clone, the PCR reaction was performed by Big Dye Terminator v3.1 (Thermo Fisher Scientific Inc.), using primers 14 (SEQ ID NO.: 63) and 15 (SEQ ID NO.: 64) for sequence confirmation, then the base sequence was analyzed by a DNA sequencer (Thermo Fisher Scientific Inc.). A vector into which the insert part was introduced, that had no variation in other part, was defined as pET17b_DED_HRV3C.

(2) Expression and Purification of DED-HRV3C

E. coli was transformed using pET17b_DED_HRV3C, and 1.5 L of an LB medium containing ampicillin was cultured at 37° C. and cooled to 15° C. around a turbidity OD of about 0.6, then IPTG was added thereto to induce expression of protein. After 16 hours, bacteria were collected by centrifugation (8,000 g, 15 minutes) and frozen at −80° C. 120 mL of a buffer (20 mM Tris-HCl (pH 8.0), 150 mM NaCl, 1 tablet Complete EDTA free (Roche)) was added to the frozen E. coli, and E. coli was crushed by ultrasonic wave. The crushed solution was centrifuged (15,000 g×30 minutes), then the supernatant fraction was filtered with 0.8 uM of a filter agent, and the filtrate was collected. The filtrate was loaded on an ion exchange resin (Hitrap Q HP (1 mL)), and purified using a mixed solution of 20 mM Tris-HCl (pH 8.0, 150 mM NaCl; buffer A) and 20 mM Tris-HCl (pH 8.0, 1000 mM NaCl; buffer B). First, only buffer A (NaCl concentration of 150 mM) was flown through the ion exchange resin (EL0), and a fraction eluted with a 3:1 mixed solution of buffer A:buffer B (NaCl concentration of 363 mM) (EL1), a fraction eluted with a 1:1 mixed solution of buffer A:buffer B (NaCl concentration of 575 mM) (EL2), a fraction eluted with a 1:3 mixed solution of buffer A:buffer B (NaCl concentration of 788 mM) (EL3), and finally, a fraction eluted with only buffer B (NaCl concentration of 1000 mM) (EL4) were each collected.

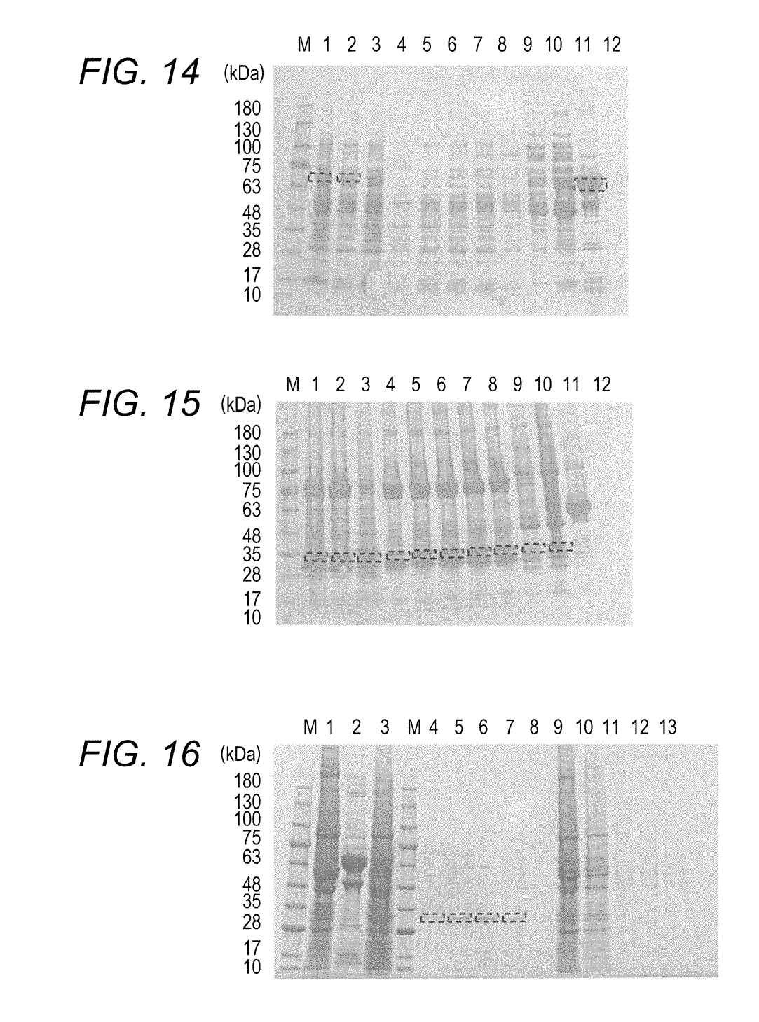

(3) The same procedures were carried out as in Example 1 to detect a fusion protein. The results were shown in FIG. 14. In FIG. 14, the results of electrophoresis are shown as follows; M: molecular weight marker, lane 1: homogenate solution, lane 2: supernatant fraction after centrifugation, lane 3: precipitated fraction after centrifugation, lanes 4 to 7: flow-through fractions 1 to 4 of the ion exchange resin, lane 8: elution fraction by buffer A (NaCl concentration of 150 mM) (EL0), lane 9: elution fraction by a 3:1 mixed solution of buffer A:buffer B (NaCl concentration of 363 mM) (EL1), lane 10: elution fraction by a 1:1 mixed solution of buffer A:buffer B (NaCl concentration of 575 mM) (EL2), lane 11: elution fraction by a 1:3 mixed solution of buffer A:buffer B (NaCl concentration of 788 mM) (EL3), and lane 12: elution fraction by buffer B (NaCl concentration of 1000 mM) (EL4).

### Comparative Example 1 [Expression and Purification of Fusion Protein DE6-HRV3Csite-NQO1 (pI=6.4)]

Peptide tag DE6 was fused to the N-terminal side of NQO1 that is a target protein, and expression and purification of a fusion protein containing HRV3Csite between NQO1 and DE6 were performed.

The same procedures were carried out as in Example 1, except for preparing an insert part using polynucleotides (SEQ ID NOs.: 66 and 67) encoding DE6 (EEEDDD; SEQ ID NO.: 65) in place of the polynucleotides encoding the peptide tag DE12, to construct a pM01_DE6 vector.

Further, pM01_DE6_HRV3Csite_NQO1 was constructed, and a recombinant baculovirus was prepared in the same manner as in Example 1, then expression, purification and detection of fusion protein DE6-HRV3Csite-NQO1 were performed. The results were shown in FIG. 15.

### Regarding Results of Examples 1 to 13 and Comparative Example 1

In Comparative Example 1 (DE6-HRV3Csite-NQO1, pI=6.4), a fusion protein was contained in every fraction, and separation was insufficient. On the other hand, Examples 1 to 13 all showed good separation. Particularly, a fusion protein binding a peptide tag with 18 or more acidic amino acid residues was held in the ion exchange resin up to a high salt concentration of 500 mM or more, and separation from contaminant proteins was improved (Examples 2 to 13). Also in all three target proteins, good separation was obtained (Examples 6 to 7 and 11 to 13). The peptide tag, even the peptide tag containing both aspartic acid residue and glutamic acid residue as acidic amino acid residues, or the peptide tag only containing either one, equally showed good separation (Examples 3 and 8). Even when the binding position of the peptide tag is either N-terminal side or C-terminal side of the target protein, separation was good (Examples 6 to 7 and 9 to 10).

### Example 14 [Separation and Detection of Target Protein NQ1 from Fusion Protein DE12-HRV3Csite-NQO1]

Protease DED-HRV3C was reacted with DE12-HRV3Csite-NQO1 to cleave DE12 from DE12-HRV3Csite-NQO1.

To Fraction EL2 collected in Example 1 was mixed a 1/10 volume of Fraction EL3 collected in Example 13, and the mixture was allowed to stand at 4° C. After 16 hours, 20 mM Tris-HCl (pH 8.0) was added to this reaction solution to be diluted to a NaCl concentration of about 250 mM, and the diluted solution was loaded on an ion exchange resin (Hitrap Q HP (1 mL)) equilibrated with 20 mM Tris-HCl (pH 8.0), 150 mM NaCl. Flow-through fractions were collected (Fr. 1 to 6 (NaCl concentration of about 250 mM)), and fractions finally eluted (Fr. 7 to 10) with only buffer B (NaCl concentration of about 1000 mM) were each collected.

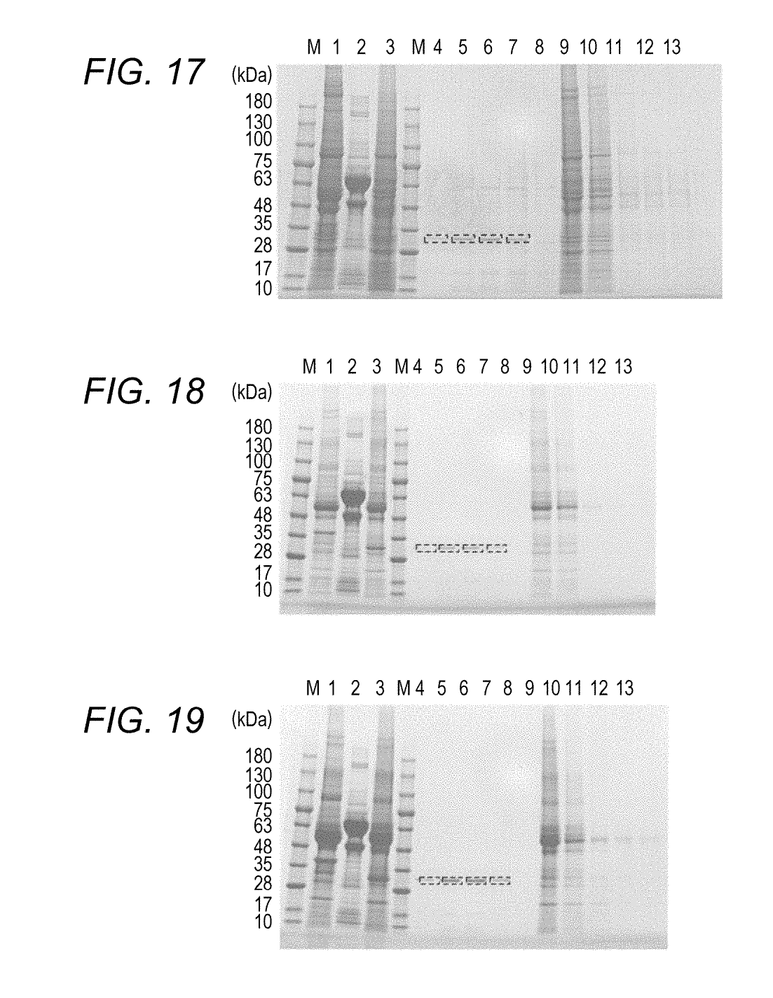

SDS-PAGE was performed using 5 to 20% gradient gel and an electrophoresis apparatus (APRO Science Inc.). Each collected fraction and β-ME Sample Treatment for Tris SDS (COSMO BIO Co., Ltd.) were mixed in a volume ratio of 1:1, and treated at 100° C. for 3 minutes, then 5 uL of the resulting solution was loaded on the gel. As a molecular weight marker, 2.5 uL of Blue Star (NIPPON Genetics Co., Ltd.) was loaded on the gel. The gel loaded with the sample was migrated at a voltage of 400 V for 14 minutes, and then the gel was stained by GelCode Blue Stain Reagent (Thermo Fisher Scientific Inc.), and excess staining was bleached, then the image was photographed using Gel Doc XR+system (Bio-Rad Laboratories, Inc.). The results were shown in FIG. 16. In FIG. 16, the results of electrophoresis are shown as follows; M: molecular weight marker, lane 1: EL2 (Examples 1 and 8) or EL3 (Examples 2 to 7 and 11 to 12), lane 2: EL3 of Example 13, lane 3: a mixed reaction liquid of EL2 or EL3 of Examples 1 to 12, and EL3 of Example 13, lanes 4 to 8: flow-through fractions 1 to 5 of the ion exchange resin, and lanes 9 to 13: elution fractions 1 to 5 by buffer B. Explanation of each lane is common in FIGS. 17 to 25.

### Example 15

The same procedures were carried out as in Example 14, except for using EL2 of Example 2 in place of EL2 of Example 1, to detect a target protein NQO1. The results were shown in FIG. 17.

### Example 16

The same procedures were carried out as in Example 14, except for using EL3 of Example 3 in place of EL2 of Example 1, to detect a target protein NQO1. The results were shown in FIG. 18.

### Example 17

The same procedures were carried out as in Example 14, except for using EL3 of Example 4 in place of EL2 of Example 1, to detect a target protein NQO1. The results were shown in FIG. 19.

### Example 18

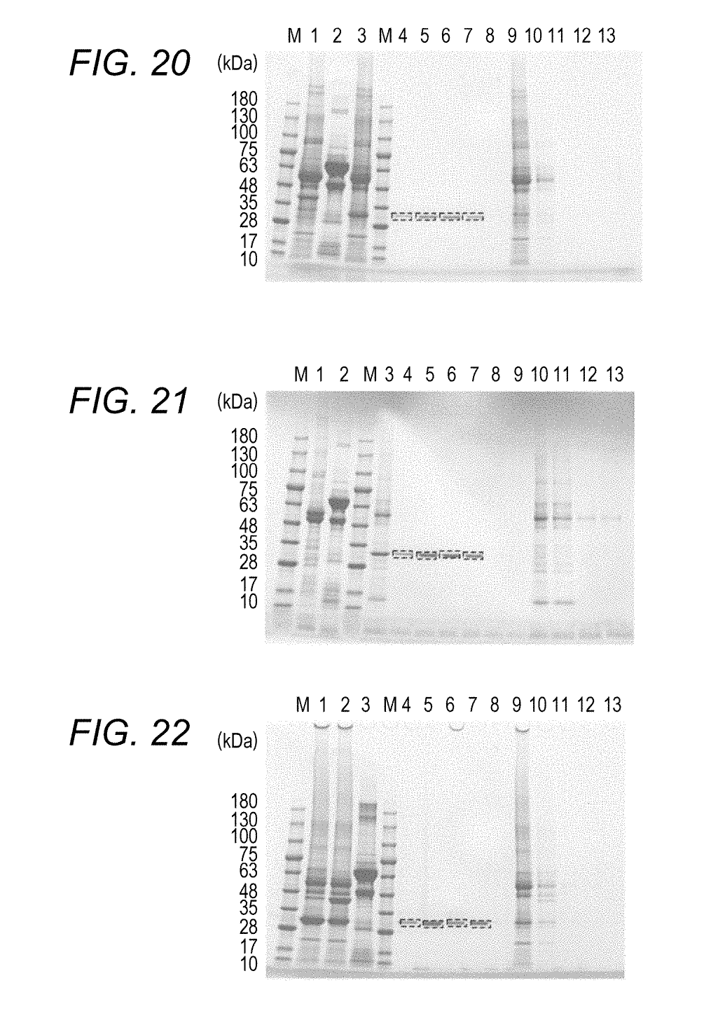

The same procedures were carried out as in Example 14, except for using EL3 of Example 5 in place of EL2 of Example 1, to detect a target protein NQO1. The results were shown in FIG. 20.

### Example 19

The same procedures were carried out as in Example 14, except for using EL3 of Example 6 in place of EL2 of Example 1, to detect a target protein NQO1. The results were shown in FIG. 21.

### Example 20

The same procedures were carried out as in Example 14, except for using EL3 of Example 7 in place of EL2 of Example 1, to detect a target protein NQO1. The results were shown in FIG. 22.

### Example 21

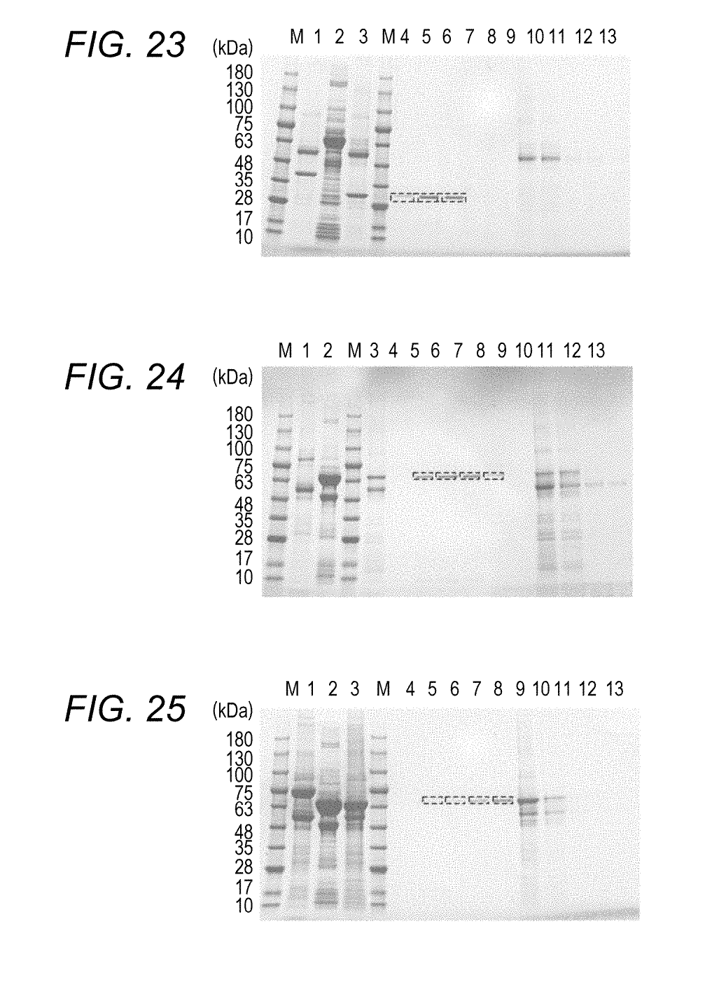

The same procedures were carried out as in Example 14, except for using EL2 of Example 8 in place of EL2 of Example 1, to detect a target protein NQO1. The results were shown in FIG. 23.

### Example 22

The same procedures were carried out as in Example 14, except for using EL3 of Example 11 in place of EL2 of Example 1, to detect a target protein Luciferase. The results were shown in FIG. 24.

### Example 23

The same procedures were carried out as in Example 14, except for using EL3 of Example 12 in place of EL2 of Example 1, to detect a target protein Luciferase. The results were shown in FIG. 25.

(Regarding Results of Examples 14 to 23)

Based on the results of Examples 14 to 23, it was shown that the protease is reacted with the fusion protein, whereby a target protein NQO1 or Luciferase can be acquired in the flow-through fraction of the anion exchange resin.

### Example 24 [Evaluation of Effect on Separation Due to pH of Buffer]

The same procedures were carried out as in Example 6 to construct pHS01_DED_HRV3Csite_NQO1.

pHS01_DED_HRV3Csite_NQO1 and baculovirus DNA were introduced into silkworm culture cells (BmN cells), and homologous recombination of virus was performed. The culture cells were cultured for 6 days, and the recombinant virus was collected. Thereafter, a virus solution diluted 50 times with MilliQ water was inoculated into pupa of silkworm, and the pupa was incubated for 6 days. The pupa of silkworm was collected, and 50 mL of a buffer (20 mM PIPES (pH 6.1), 150 mM NaCl, 1 tablet Complete EDTA free (Roche), phenylthiourea) was added per a pupa to homogenate the pupa. The solution was centrifuged (8,000 g, 10 minutes), then the supernatant fraction was filtered with 0.8 uM of a filter agent, and the filtrate was collected. The filtrate was loaded on an ion exchange resin (Hitrap Q HP (1 mL)), and purified using a mixed solution of 20 mM PIPES (pH 6.1, 150 mM NaCl; buffer C) and 20 mM PIPES (pH 6.1, 1000 mM NaCl; buffer D). First, only buffer C (NaCl concentration of 150 mM) was flown through the ion exchange resin (EL0), and a fraction eluted with a solution with a mixing ratio of buffer C:buffer D of 3:1 (NaCl concentration of 363 mM) (EL1), a fraction eluted with a solution with a mixing ratio of buffer C:buffer D of 1:1 (NaCl concentration of 575 mM) (EL2), a fraction eluted with a solution with a mixing ratio of buffer C:buffer D of 1:3 (NaCl concentration of 788 mM) (EL3), and finally, a fraction eluted with only buffer D (NaCl concentration of 1000 mM) (EL4) were each collected.

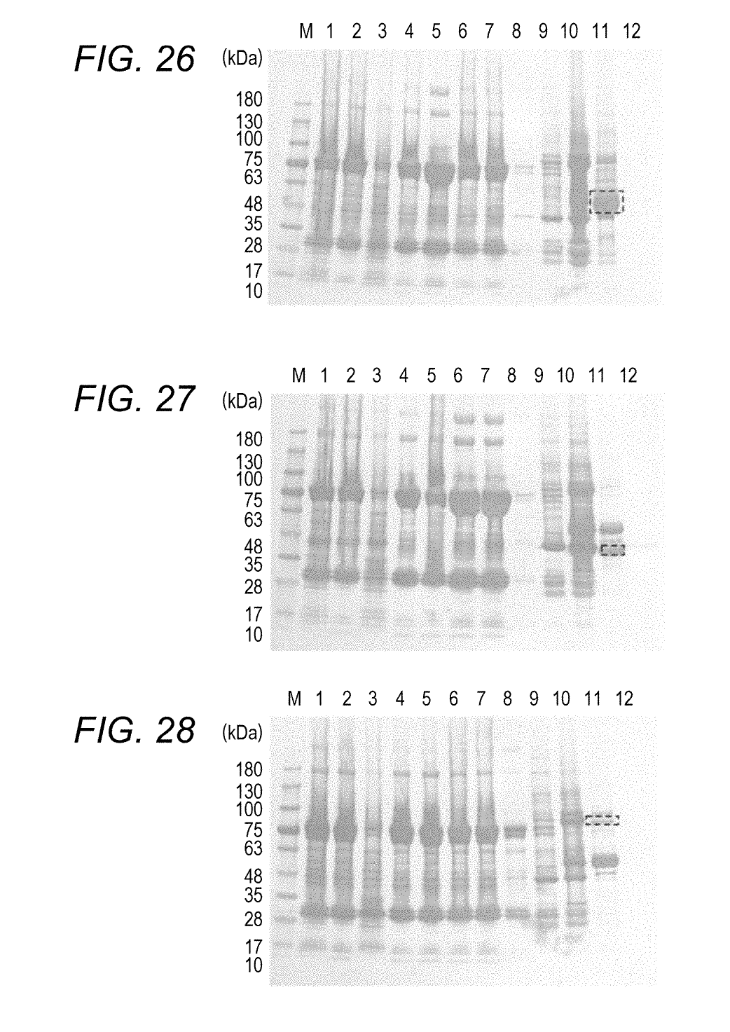

The same procedures were carried out as in Example 1 to detect DED-HRV3Csite-NQO1. The results were shown in FIG. 26. In FIG. 26, the results of electrophoresis are shown as follows; M: molecular weight marker, lane 1: homogenate solution, lane 2: supernatant fraction after centrifugation, lane 3: precipitated fraction after centrifugation, lanes 4 to 7: flow-through fractions 1 to 4 of the ion exchange resin, lane 8: elution fraction by buffer C (NaCl concentration of 150 mM) (EL0), lane 9: elution fraction by a 3:1 mixed solution of buffer C:buffer D (NaCl concentration of 363 mM) (EL1), lane 10: elution fraction by a 1:1 mixed solution of buffer C:buffer D (NaCl concentration of 575 mM) (EL2), lane 11: elution fraction by a 1:3 mixed solution of buffer C:buffer D (NaCl concentration of 788 mM) (EL3), and lane 12: elution fraction by buffer D (NaCl concentration of 1000 mM) (EL4). Explanation of each lane is common in FIGS. 27 to 28.

### Example 25

The same procedures were carried out as in Example 7 to construct pHS01_DES_HRV3Csite_NQO1.

Furthermore, the same procedures were carried out as in Example 24 to express, purify and detect DES-HRV3Csite-NQO1. The results were shown in FIG. 27.

### Example 26

The same procedures were carried out as in Example 11 to construct pHS01_DED_HRV3Csite_Luciferase.

Furthermore, the same procedures were carried out as in Example 24 to express, purify and detect DED-HRV3Csite-Luciferase. The results were shown in FIG. 28.

### Example 27

The same procedures were carried out as in Example 7 to construct pHS01_DES_HRV3Csite_NQO1.

E. coli was transformed using pHS01_DES_HRV3Csite_NQO1, and 1.5 L of an LB medium containing ampicillin was cultured at 37° C. and cooled to 15° C. around a turbidity OD of about 0.6, then IPTG was added thereto to induce expression of protein. After 16 hours, bacteria were collected by centrifugation (8,000 g, 15 minutes) and frozen at −80° C. 120 mL of a buffer (20 mM PIPES (pH 6.1), 150 mM NaCl, 1 tablet Complete EDTA free (Roche)) was added to the frozen E. coli, and E. coli was crushed by ultrasonic wave. The crushed solution was centrifuged (15,000 g×30 minutes), then the supernatant fraction was filtered with 0.8 uM of a filter agent, and the filtrate was collected. The filtrate was loaded on an ion exchange resin (Hitrap Q HP (1 mL)), and purified using a mixed solution of 20 mM PIPES (pH 6.1, 150 mM NaCl; buffer C) and 20 mM PIPES (pH 6.1, 1000 mM NaCl; buffer D). First, only buffer C (NaCl concentration of 150 mM) was flown through the ion exchange resin (EL0), and a fraction eluted with a 3:1 mixed solution of buffer C:buffer D (NaCl concentration of 363 mM) (EL1), a fraction eluted with a 1:1 mixed solution of buffer C:buffer D (NaCl concentration of 575 mM) (EL2), a fraction eluted with a 1:3 mixed solution of buffer C:buffer D (NaCl concentration of 788 mM) (EL3), and finally, a fraction eluted with only buffer D (NaCl concentration of 1000 mM) (EL4) were each collected.

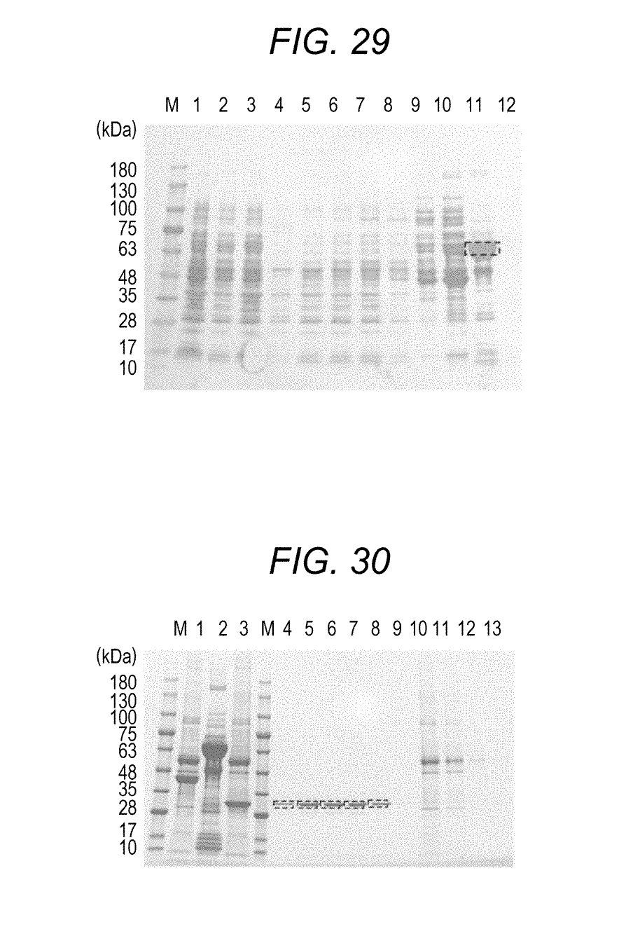

The same procedures were carried out as in Example 1 to detect DED-HRV3Csite-NQO1. The results were shown in FIG. 29. In FIG. 29, the results of electrophoresis are shown as follows; M: molecular weight marker, lane 1: homogenate solution, lane 2: supernatant fraction after centrifugation, lane 3: precipitated fraction after centrifugation, lanes 4 to 7: flow-through fractions 1 to 4 of the ion exchange resin, lane 8: elution fraction by buffer C (NaCl concentration of 150 mM) (EL0), lane 9: elution fraction by a 3:1 mixed solution of buffer C:buffer D (NaCl concentration of 363 mM) (EL1), lane 10: elution fraction by a 1:1 mixed solution of buffer C:buffer D (NaCl concentration of 575 mM) (EL2), lane 11: elution fraction by a 1:3 mixed solution of buffer C:buffer D (NaCl concentration of 788 mM) (EL3), and lane 12: elution fraction by buffer D (NaCl concentration of 1000 mM; EL4).

### Example 28

To EL3 of Example 24 was added a 1/10 volume of EL3 of Example 27, and the mixture was allowed to stand at 4° C. After 16 hours, 20 mM PIPES (pH 6.1) was added to the reaction solution to be diluted to a NaCl concentration of about 250 mM, and the diluted solution was loaded on an ion exchange resin (Hitrap Q HP (1 mL)) equilibrated with 20 mM PIPES (pH 6.1, 150 mM NaCl). Flow-through fractions were collected (Fr. 1 to 6 (NaCl concentration of about 250 mM)), and fractions finally eluted (Fr. 7 to 10) with only buffer D (NaCl concentration of about 1000 mM) were each collected.

The same procedures were carried out as in Example 1 to detect a target protein EQO1. The results were shown in FIG. 30. In FIG. 30, the results of electrophoresis are shown as follows; M: molecular weight marker, lane 1: EL3 of Example 24, lane 2: EL3 of Example 27, lane 3: a mixed reaction liquid of EL3 of Example 24, and EL3 of Example 27, lanes 4 to 8: flow-through fractions 1 to 5 of the ion exchange resin, and lanes 9 to 13: elution fractions 1 to 5 by buffer D.

### Regarding Results of Examples 24 to 28

Based on the results of Examples 24 to 28, it was shown that, in the expression and purification of the fusion protein and purification of the target protein, good separation is obtained even the pH of the buffer is lowered.

1. A method for purifying a protein, comprising steps of:
preparing a sample comprising a fusion protein and contaminant proteins, the fusion protein comprising an amino acid sequence of a peptide tag and an amino acid sequence of a target protein, and
separating the contaminant proteins and the fusion protein,
wherein the peptide tag comprises 12 or more acidic amino acid residues.

2. The method for purifying a protein according to claim 1, wherein the peptide tag comprises any of the amino acid sequences of SEQ ID NOs. 1 to 8.

3. The method for purifying a protein according to claim 1, wherein the peptide tag comprises 18 or more acidic amino acid residues.

4. The method for purifying a protein according to claim 1, wherein the isoelectric point of the fusion protein is less than 6.

5. The method for purifying a protein according to claim 1, wherein the salt concentration of the sample is 50 mM or more and 500 mM or less.

6. The method for purifying a protein according to claim 1, wherein, in the sample preparation step,
a vector containing a polynucleotide encoding the fusion protein is introduced into a host cell, and the protein is expressed in the host cell to prepare the sample.

7. The method for purifying a protein according to claim 1, wherein the fusion protein further comprises a cleavable site recognized by a protease, between the amino acid sequence of a peptide tag and the amino acid sequence of the target protein.

8. The method for purifying a protein according to claim 1, wherein, in the step of separating the contaminant proteins, the fusion protein and the contaminant proteins are separated using an ion exchange resin.

9. The method for purifying a protein according to claim 8, wherein the ion exchange resin is an anion exchange resin.

10. The method for purifying a protein according to claim 1, wherein, the step of separating the contaminant proteins comprises
passing the sample through a anion exchange resin to obtain (i) the anion exchange resin to which the fusion protein is bound and (ii) a flow-through fraction comprising the contaminant proteins, and
eluting the fusion protein from the anion exchange resin using a buffer with a salt concentration of 600 mM or more.

11. The method for purifying a protein according to claim 1, after the step of separating the contaminant proteins,
further comprising a step of
separating the fusion protein into the target protein and the peptide tag in a solution, using the protease recognizing the cleavable site.

12. The method for purifying a protein according to claim 11, wherein, in the step of separating the fusion protein,
the solution comprising the separated target protein and the separated peptide tag is passed through an anion exchange resin, and
(i) the anion exchange resin to which the separated peptide tag is bound, and (ii) a flow-through fraction comprising the target protein are obtained.

13. The method for purifying a protein according to claim 10, wherein the salt concentration of the solution comprising the target protein and the peptide tag is 500 mM or less.

14. A method for purifying a protein, comprising steps of:
preparing a sample comprising a fusion protein and contaminant proteins, the fusion protein comprising an amino acid sequence of a peptide tag and an amino acid sequence of a target protein,
passing the sample through an anion exchange resin to obtain (i) the anion exchange resin to which the fusion protein is bound and (ii) a flow-through fraction comprising the contaminant proteins,
removing the flow-through fraction, and
passing a buffer through the anion exchange resin to elute the fusion protein from the anion exchange resin, a salt concentration of the buffer being 600 mM or more.

15. The method for purifying a protein according to claim 14, wherein the peptide tag comprises any of the amino acid sequences of SEQ ID NOs. 1 to 8.

16. The method for purifying a protein according to claim 14, wherein the peptide tag comprises 18 or more acidic amino acid residues.

17. The method for purifying a protein according to claim 14, wherein the isoelectric point of the fusion protein is less than 6.

18. The method for purifying a protein according to claim 14, wherein the salt concentration of the sample is 50 mM or more and 500 mM or less.

19. The method for purifying a protein according to claim 14, wherein, in the sample preparation step,
a vector containing a polynucleotide encoding the fusion protein is introduced into a host cell, and the protein is expressed in the host cell to prepare the sample.

20. A method for purifying a protein, comprising steps of:
preparing a sample comprising a fusion protein and contaminant proteins, the fusion protein comprising an amino acid sequence of a peptide tag, an amino acid sequence of a target protein and a cleavable site recognized by a protease, the cleavable site being located between the amino acid sequence of the peptide tag and the amino acid sequence of the target protein,
passing the sample through an anion exchange resin to obtain (i) the anion exchange resin to which the fusion protein is bound and (ii) a flow-through fraction comprising the contaminant proteins,
removing the flow-through fraction,
passing a buffer through the anion exchange resin to elute the fusion protein from the anion exchange resin to obtain a flow-through fraction comprising the fusion protein, a salt concentration of the buffer being 600 mM or more,
mixing a protease recognizing the cleavable site and the flow-through fraction comprising the fusion protein to obtain a solution comprising a separated target protein and a separated peptide tag, and
passing the solution comprising the separated target protein and the separated peptide tag through an anion exchange resin to obtain (i) the anion exchange resin to which the separated peptide tag is bound, and (ii) a flow-through fraction comprising the separated target protein.
